(19) United States
(12) Patent Application Publication
(10) Pub. No.: US 2019/0308934 A1
(43) Pub. Date: Oct. 10, 2019

(54) Prodrugs of Chlorokynurenines

(71) Applicant: CEPHALON, INC., Frazer, PA (US)

(72) Inventors: Ralph Laufer, Tel Aviv (IL); Gregory R. Ott, Media, PA (US)

(21) Appl. No.: 16/449,612

(22) Filed: Jun. 24, 2019

Related U.S. Application Data
(63) Continuation of application No. 15/756,692, filed on Mar. 1, 2018, now abandoned.
(60) Provisional application No. 62/215,276, filed on Sep. 8, 2015.

(51) Int. Cl. C07C 271/22; C07C 233/05; C07C 271/28; C07C 305/12; C07D 207/09; C07D 223/16; C07D 263/18; C07F 9/09; A61K 31/165; A61K 31/18; A61K 31/196; A61K 31/198; A61K 31/216; A61K 31/24; A61K 31/255; A61K 31/325; A61K 31/40; A61K 31/421; A61K 31/55; A61K 31/661; A61K 31/7028; A61K 38/03; C07K 4/00; C07C 233/36; C07D 207/08; C07H 13/04; C07D 223/12; C07C 237/20; C07C 233/54; C07C 233/51; C07C 229/42; C07C 311/51; C07C 233/47

(52) U.S. Cl. CPC C07C 271/22; C07C 233/47; C07C 271/28; C07C 305/12; C07D 207/09; C07D 223/16; C07D 263/18; C07F 9/09; A61K 31/165; A61K 31/18; A61K 31/196; A61K 31/198; A61K 31/216; A61K 31/24; A61K 31/255; A61K 31/325; A61K 31/40; A61K 31/421; A61K 31/55; A61K 31/661; A61K 31/7028; A61K 38/03; C07K 4/00; C07C 233/36; C07D 207/08; C07H 13/04; C07D 223/12; C07C 237/20; C07C 233/54; C07C 233/51; C07C 229/42; C07C 311/51; C07C 233/05

(57) ABSTRACT

The present disclosure relates to prodrugs of 7-chlorokynurenic acid. In certain embodiments, the prodrugs include those having the structure of any one of formula (I)-(VIII), wherein R¹-R¹³, monomer 1, monomer 2, and linker are defined herein. Also provided are methods of preparing and using these prodrugs.

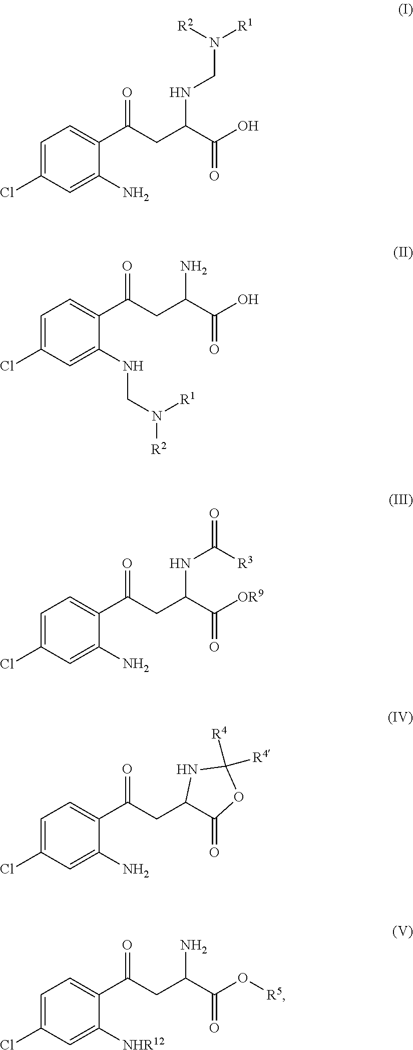

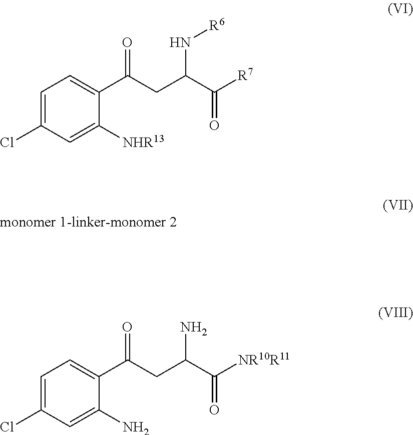

### CROSS-REFERENCE TO RELATED APPLICATIONS

This application is a continuation of U.S. patent application Ser. No. 15/756,692, filed Mar. 1, 2018, which is the National Stage Application of International Patent Application No. PCT/US2016/050602, filed Sep. 8, 2016, which claims the benefit of U.S. Provisional Patent Application No. 62/215,276, filed Sep. 8, 2015, which are incorporated by reference herein.

### TECHNICAL FIELD

The disclosure is in the field of kynurenine prodrugs and methods of their use.

### BACKGROUND

Many currently-approved antidepressants, such as selective serotonin reuptake inhibitors and serotonin norepinephrine reuptake inhibitors, have limited effectiveness due to their mechanism of action. It is often necessary for patients to take such medications for weeks prior to experiencing a benefit. The mechanism of action for 7-chlorokynurenic acid differs from other antidepressants since it targets glycine site of the N-methyl-D-aspartate (NMDA) receptor. Accordingly, it has the potential to effectively to treat patients who do not respond to antidepressants that do not act from the NMDA receptor. Unfortunately, 7-chlorokynurenic acid does not cross the blood-brain barrier and, therefore, cannot be used as a therapeutic agent.

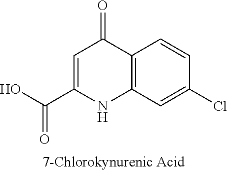 [SMILES: O=C(O)c1cc(=O)c2ccc(Cl)cc2[nH]1]

4-Chlorokynurenine converts into 7-chlorokynurenic acid in vivo and has the advantage of crossing the blood-brain barrier. Accordingly, it is a potent and selective NMDA antagonist and down-regulates the NMDA receptor. It may be synthesized as described in U.S. Pat. No. 5,547,991 and Salituro “Enzyme-Activated Antagonists of the Strychnine-Insensitive Glycine/NMDA Receptor, J. Med. Chem. 1994; 37-334,336. L-4-chlorokynurenine is also commercially available commercially from various sources.

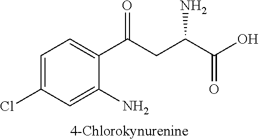 [SMILES: Nc1cc(Cl)ccc1C(=O)C[C@H](N)C(=O)O]

Thus, the development and evaluation of 7-chlorokynurenic acid prodrugs is highly desirable so as to identify alternative and potentially improved clinical candidates. This disclosure is directed to these and other important needs.

### SUMMARY

In certain embodiments, compounds having the structure of formula (I) or (II), or a pharmaceutically acceptable salt, stable isotope, or stereoisomer thereof, are provided, wherein R¹ and R² are defined herein.

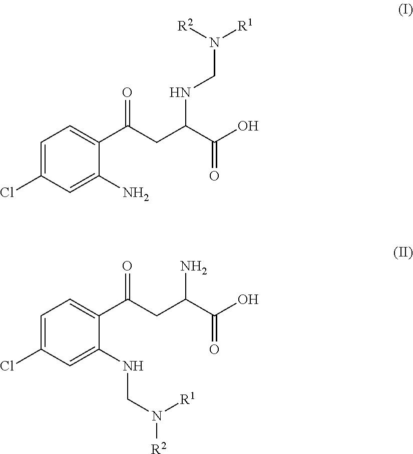

In other embodiments, compounds having the structure of formula (III), or a pharmaceutically acceptable salt, stable isotope, or stereoisomer thereof, are provided, wherein R³ and R⁹ are defined herein.

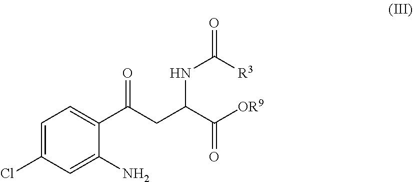

In some embodiments, compounds having the structure of formula (IV), or a pharmaceutically acceptable salt, stable isotope, or stereoisomer thereof, are provided, wherein R⁴ and R^{4′} are defined herein.

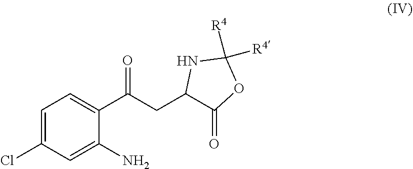

In other embodiments, compounds having the structure of formula (V), or a pharmaceutically acceptable salt, stable isotope, or stereoisomer thereof, are provided, wherein R⁵ and R¹² are defined herein.

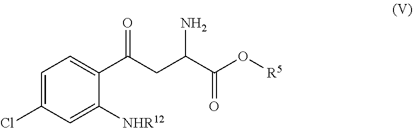

In further embodiments, compounds having the structure of formula (VI), or a pharmaceutically acceptable salt, stable isotope, or stereoisomer thereof, are provided, wherein R⁶ and R⁷ are defined herein.

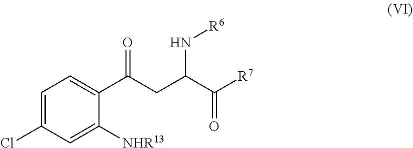

In some embodiments, compounds having the structure of formula (VII), or a pharmaceutically acceptable salt, stable isotope, or stereoisomer thereof, are provided wherein monomer 1 and monomer 2 are, independently, the structure of formula (I), (II), or (III) and R¹-R³ are defined herein.

monomer 1-linker-monomer 2 (VII)

In further embodiments, compounds having the structure of formula (VIII), or a pharmaceutically acceptable salt, stable isotope, or stereoisomer thereof, are provided, wherein R¹⁰ and R¹¹ are defined herein.

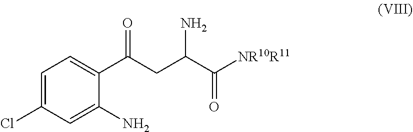

Methods of using the described compounds are also disclosed.

### DETAILED DESCRIPTION OF ILLUSTRATIVE EMBODIMENTS

The present disclosure may be understood more readily by reference to the following detailed description taken in connection with the accompanying figures and examples, which form a part of this disclosure. It is to be understood that this disclosure is not limited to the specific compositions or methods described and/or shown herein, and that the terminology used herein is for the purpose of describing particular embodiments by way of example only and is not intended to be limiting of the claimed disclosure. Also, as used in the specification including the appended claims, the singular forms “a,” “an,” and “the” include the plural, and reference to a particular numerical value includes at least that particular value, unless the context clearly dictates otherwise. Similarly, when values are expressed as approximations, by use of the antecedent “about,” it will be understood that the particular value forms another embodiment. All ranges are inclusive and combinable.

It is to be appreciated that certain features of the disclosure which are, for clarity, described herein in the context of separate embodiments, may also be provided in combination in a single embodiment. Conversely, various features of the disclosure that are, for brevity, described in the context of a single embodiment, may also be provided separately or in any subcombination. Further, reference to values stated in ranges includes each and every value within that range.

As used herein, the term “substituted” refers to where at least one hydrogen atom of a chemical group is replaced by a non-hydrogen moiety. In certain embodiments, the substituents include, without limitation, OH, oxo, C(O)OH, C₁₋₆ alkyl, C₁₋₆ alkoxy, amino, halogen, C₁₋₆ haloalkyl, C₃₋₈ cycloalkyl, OC(O)C₁₋₆ alkyl, C(O)aryl, C(O)C₁₋₆ alkoxy, aryl, heteroaryl, or heterocyclyl. The C₃₋₈ cycloalkyl, aryl, heteroaryl, or heterocyclyl groups may, themselves, be optionally substituted.

“Alkyl” refers to a monoradical of a branched or unbranched saturated hydrocarbon chain. In certain embodiments, an alkyl is, without limitation, methyl, ethyl, n-propyl, n-butyl, n-pentyl, n-hexyl, n-heptyl, n-octyl, n-nonyl, n-decyl, isopropyl, tert-butyl, isobutyl, etc. Alkyl groups may contain 1 to about 10 carbon atoms, such as 1 to about 6 carbon atoms or 1 to about 4 carbon atoms, and can be substituted or unsubstituted.

“Amino” refers to a NH₂, NH(C₁₋₆ alkyl), or N(C₁₋₆ alkyl)(C₁₋₆ alkyl), wherein the alkyl groups are, independently, optionally substituted as described above.

“Arylalkyleneoxyl” refers to a mono radical of an aryl moiety bound to a branched or unbranched saturated hydrocarbon chain bound to an O-atom. Alkylene groups may contain 1-10 carbon atoms, such as 1-6 carbon atoms, and can be substituted or unsubstituted. Examples include, but are not limited to, methylene (—OCH₂—), the ethylene isomers (—OCH(CH₃)— and —OCH₂CH₂—), the propylene isomers (—OCH(CH₃)CH₂—, —OCH(CH₂CH₃)—, —OC(CH₃)₂—, and —OCH₂CH₂CH₂—), etc.

“Alkylene glycol” refers to a moiety of the structure —(OCₙH₂ₙ)ₚ—OCₙH₂ₙ₊₁, wherein n is 1 to about 10 and p is 1 to about 20. In certain embodiments, the alkylene glycol is —OCH(CH₃)—O—CH(CH₃)₂ or —OC(CH₃)₂O—CH(CH₃)₂.

“Alkoxy” as used herein refers to the O-(alkyl) group, where the point of attachment is through the oxygen-atom and the alkyl group is defined herein.

“Cycloalkyl” refers to a monoradical non-aromatic carbocyclic ring system, which may be saturated or unsaturated, substituted or unsubstituted, and may be monocyclic, bicyclic, or tricyclic, and may be bridged, spiro, and/or fused. The cycloalkyl group may contain from 3 to about 10 ring atoms, such as 3 to about 7 ring atoms, 3 ring atoms, 5 ring atoms, 6 ring atoms, or 7 ring atoms. In certain embodiments, a cycloalkyl includes, but are not limited to, cyclopropyl, cyclobutyl, cyclopentyl, cyclohexyl, norbomyl, bicyclo[2.2.1]hexane, bicyclo[2.2.1]heptane, bicyclo[3.1.1]heptane, bicyclo[3.2.1]octane, bicyclo[2.2.2]octane, bicyclo[3.2.2]nonane, bicyclo[3.3.1]nonane, and bicyclo[3.3.2]decane.

“Aryl” refers to phenyl and 7-15 membered monoradical bicyclic or tricyclic hydrocarbon ring systems, including bridged, spiro, and/or fused ring systems, in which at least one of the rings is aromatic. Aryl groups can be substituted or unsubstituted. An aryl group may contain 6 (i.e., phenyl) or about 9 to about 15 ring atoms, such as 6 (i.e., phenyl) or about 9 to about 11 ring atoms. In some embodiments, aryl groups include, but are not limited to, naphthyl, indanyl, indenyl, anthryl, phenanthryl, fluorenyl, 1,2,3,4-tetrahydronaphthalenyl, 6,7,8,9-tetrahydro-5H-benzocycloheptenyl, and 6,7,8,9-tetrahydro-5H-benzocycloheptenyl.

“Haloalkyl” refers to alkyl groups in which one or more hydrogen atom is replaced by a halogen atom. Haloalkyl includes alkyl groups, such as CF₃, CHF₂, CH₂F, CF₂CF₃, CHFCF₃, CH₂CF₃, CF₂CH₃, CHFCH₃, CF₂CF₂CF₃, and CF₂CH₂CH₃.

“Halogen” includes fluorine, chlorine, bromine and iodine atoms.

“Heteroaryl” refers to (a) 5 and 6 membered monocyclic aromatic rings, which contain, in addition to carbon atoms, at least one heteroatom, such as nitrogen, oxygen or sulfur, and (b) 7-15 membered bicyclic and tricyclic rings, which contain, in addition to carbon atoms, at least one heteroatom, such as nitrogen, oxygen or sulfur, and in which at least one ring is aromatic. Heteroaryl groups can be substituted or unsubstituted, and may be bridged, spiro, and/or fused. A heteroaryl may contain at least about 5 ring atoms. In further embodiments, a heteroaryl may contain 5 to about 15 ring atoms. In further embodiments, a heteroaryl may contain 5 to about 10 ring atoms, such as 5, 6, 9, or 10 ring atoms. Unless otherwise indicated, the foregoing heteroaryls can be C-attached or N-attached where such is possible and results in the creation of a stable structure. In certain embodiments, heteroaryl includes, but is not limited to, 2,3-dihydrobenzofuranyl, 1,2-dihydroquinolinyl, 3,4-dihydroisoquinolinyl, 1,2,3,4-tetrahydro-isoquinolinyl, 1,2,3,4-tetrahydroquinolinyl, benzoxazinyl, benzthiazinyl, chromanyl, furanyl, imidazolyl, isoxazolyl, isothiazolyl, oxadiazolyl, oxazolyl, pyridinyl, pyrimidinyl, pyrazolyl, pyrrolyl, pyrazinyl, pyridazinyl, pyrazinyl, thienyl, tetrazolyl, thiazolyl, thiadiazolyl, triazinyl, triazolyl, naphthyridinyl, pteridinyl, phthalazinyl, purinyl, alloxazinyl, benzimidazolyl, benzofuranyl, benzofurazanyl, 2H-1-benzopyranyl, benzothiadiazinyl, benzothiazinyl, benzo-thiazolyl, benzothiophenyl, benzoxazolyl, cinnolinyl, furopyridinyl, indolinyl, indolizinyl, indolyl, quinazolinyl, quinoxalinyl, isoindolyl, isoquinolinyl, 10-aza-tricyclo[6.3.1.0^{2,7}]dodeca-2(7),3,5-trienyl, 12-oxa-10-aza-tricyclo[6.3.1.0^{2,7}]dodeca-2(7),3,5-trienyl, 12-aza-tricyclo-[7.2.1.0^{2,7}]dodeca-2(7),3,5-trienyl, 10-aza-tricyclo[6.3.2.0^{2,7}]trideca-2(7),3,5-trienyl, 2,3,4,5-tetrahydro-1H-benzo[d]azepinyl, 1,3,4,5-tetrahydro-benzo[d]azepin-2-onyl, 1,3,4,5-tetrahydro-benzo[b]azepin-2-onyl, 2,3,4,5-tetrahydro-benzo[c]azepin-1-onyl, 1,2,3,4-tetrahydro-benzo[e][1,4]diazepin-5-onyl, 2,3,4,5-tetrahydro-1H-benzo[e][1,4]diazepinyl, 5,6,8,9-tetrahydro-7-oxa-benzocycloheptenyl, 2,3,4,5-tetrahydro-1H-benzo[b]azepinyl, 1,2,4,5-tetrahydro-benzo-[e][1,3]diazepin-3-onyl, 3,4-dihydro-2H-benzo[b][1,4]dioxepinyl, 3,4-dihydro-2H-benzo[f][1,4]-oxazepin-5-onyl, 6,7,8,9-tetrahydro-5-thia-8-aza-benzocycloheptenyl, 5,5-dioxo-6,7,8,9-tetrahydro-5-thia-8-aza-benzocycloheptenyl, and 2,3,4,5-tetrahydro-benzo[f][1,4]oxazepinyl.

“Heterocycle” refers to 3-15 membered monocyclic, bicyclic, and tricyclic non-aromatic rings, which may be saturated or unsaturated, can be substituted or unsubstituted, may be bridged, spiro, and/or fused, and which contain, in addition to carbon atoms, at least one heteroatom, such as nitrogen, oxygen, sulfur or phosphorus. A heterocycle may contain, in addition to carbon atoms, at least one nitrogen, oxygen, or sulfur. A heterocycle may contain from 3 to about 10 ring atoms, 3 to about 7 ring atoms, 5 to 7 ring atoms, 5 ring atoms, 6 ring atoms, or 7 ring atoms. Unless otherwise indicated, the foregoing heterocycles can be C-attached or N-attached where such is possible and results in the creation of a stable structure. Examples include, but are not limited to, tetrahydrofuranyl, pyrrolidinyl, pyrrolinyl, imidazolidinyl, imidazolinyl, azetidinyl, pyrazolidinyl, pyrazolinyl, piperidinyl, piperazinyl, indolinyl, isoindolinyl, morpholinyl, thiomorpholinyl, homomorpholinyl, homopiperidinyl, homopiperazinyl, thiomorpholinyl-5-oxide, thiomorpholinyl-S,S-dioxide, tetrahydropyranyl, piperidinyl, tetrahydrothienyl, homothiomorpholinyl-S,S-dioxide, oxazolidinonyl, dihydropyrazolyl, dihydropyrazinyl, dihydropyridinyl, dihydropyrimidinyl, dihydrofuryl, dihydropyranyl, tetrahydrothienyl-5-oxide, tetrahydrothienyl-S,S-dioxide, homothiomorpholinyl-5-oxide, quinuclidinyl, 2-oxa-5-azabicyclo[2.2.1]heptanyl, 8-oxa-3-aza-bicyclo[3.2.1]octanyl, 3,8-diaza-bicyclo[3.2.1]octanyl, 2,5-diaza-bicyclo[2.2.1]heptanyl, 3,8-diaza-bicyclo[3.2.1]-octanyl, 3,9-diaza-bicyclo[4.2.1]nonanyl, 2,6-diaza-bicyclo[3.2.2]nonanyl, [1,4]oxaphos-phinanyl-4-oxide, [1,4]azaphosphinanyl-4-oxide, [1,2]oxaphospholanyl-2-oxide, phosphinanyl-1-oxide, [1,3]azaphospholidinynl-3-oxide, [1,3]oxaphospholanyl-3-oxide and 7-oxabicyclo[2.2.1]heptanyl.

“Amino acid” as used herein refers to the standard and non-standard amino acids known in the art. In certain embodiments, the amino acid is a standard amino acid such as alanine, arginine, asparagine, aspartic acid, cysteine, glutamic acid, glutamine, glycine, histidine, isoleucine, leucine, lysine, methionine, phenylalanine, proline, serine, threonine, tryptophan, tyrosine, and valine. In other embodiments, the amino acid is a non-standard amino acid such as selenocysteine, pyrrolysine, and N-formylmethionine.

“Pharmaceutically acceptable” refers to physiologically tolerable materials, which do not typically produce an allergic or other untoward reaction when administered to a human.

“Pharmaceutical composition” refers to a composition that can be used to treat a disease, condition, or disorder in a human.

“Therapeutically effective amount” refers to an amount of a compound described herein which is sufficient to inhibit, halt, or cause an improvement in a disorder or condition being treated in a particular subject or subject population. In certain embodiments, in a human or other mammal, a therapeutically effective amount can be determined experimentally in a laboratory or clinical setting, or may be the amount required by government guidelines for the particular disease and subject being treated. In other embodiments, the therapeutically effective amount is the amount of the chlorokynurenine prodrug described herein which is effective to down-regulate a NMDA receptor mediated signal transmission. It should be appreciated that determination of proper dosage forms, dosage amounts, and routes of administration is within the level of ordinary skill in the pharmaceutical and medical arts.

“Treatment” refers to the acute or prophylactic diminishment or alleviation of at least one symptom or characteristic associated or caused by a disorder being treated. In certain embodiments, treatment can include diminishment of several symptoms of a disorder or complete eradication of a disorder.

As used herein, “patient” or “subject” is intended to mean a mammal. Thus, the methods described herein are applicable to human and nonhuman subjects. In certain embodiments, the methods described herein are applicable to humans. It should be understood that the subject to be treated as described herein is in recognized need of such treatment.

The subject disclosure is also intended to include all isotopes of atoms occurring on the compounds disclosed herein. Isotopes include those atoms having the same atomic number but different mass numbers. By way of general example and without limitation, Isotopes of hydrogen include tritium and deuterium. Isotopes of carbon include C-13 and C-14. Isotopes of nitrogen include N-14 and N-15.

It will also be noted that any notation of a hydrogen in structures throughout this application, when used without further notation, are intended to represent all isotopes of hydrogen, such as ¹H, ²H, or ³H. Furthermore, any compounds containing ²H or ³H may specifically have the structure of any of the compounds disclosed herein.

It will be noted that any notation of a carbon in structures throughout this application, when used without further notation, are intended to represent all isotopes of carbon, such as ¹²C, ¹³C, or ¹⁴C. Furthermore, any compounds containing ¹³C or ¹⁴C may specifically have the structure of any of the compounds disclosed herein.

It will be noted that any notation of a nitrogen in structures throughout this application, when used without further notation, are intended to represent all isotopes of nitrogen, such as ¹⁴N or ¹⁵N. Furthermore, any compounds containing ¹⁴N or ¹⁵N may specifically have the structure of any of the compounds disclosed herein.

As used herein, an “isotopically-enriched” compound means that the abundance of deuterium, ¹³C, or ¹⁵N at any relevant site of the compound is more than the abundance of deuterium, ¹³C, or ¹⁵N naturally occurring at that site in an amount of the compound. A relevant site in a compound as used above is a site which would be designated as “H” or “C” or “N” in a chemical structure representation of the compound when not enriched. “Naturally occurring” as used above refers to the abundance of the particular atom which would be present at a relevant site in a compound if the compound was prepared without any affirmative step to enrich the abundance of the isotope. Thus, for example in a “deuterium-enriched” compound, the abundance of deuterium at any of its relevant sites can range from more than 0.0156% to 100%. Examples of ways to obtain a deuterium-enriched compound are exchanging hydrogen with deuterium or synthesizing the compound with deuterium-enriched starting materials.

Isotopically-labeled compounds can generally be prepared by conventional techniques known to those skilled in the art or by processes analogous to those described in the Examples disclosed herein using an appropriate isotopically-labeled reagents in place of the non-labeled reagents employed.

The compounds of formulas (I), (II), (III), (IV), (V), (VI), (VII), and (VIII) will convert to 4-chlorokynurenine after administration to a patient, for example, a human. In some embodiments, the compounds of formulas (I), (II), (III), (IV), (V), (VI), (VII), and (VIII) will convert to 7-chlorokynurenic acid after administration to a patient, for example, a human.

In certain embodiments, compounds having the structure of formula (I) or (II) are provided. Enantiomers of the compounds of formula (I) and/or (II) are also contemplated. In certain embodiments, the compound has the structure of the formula (IA) or (IIA).

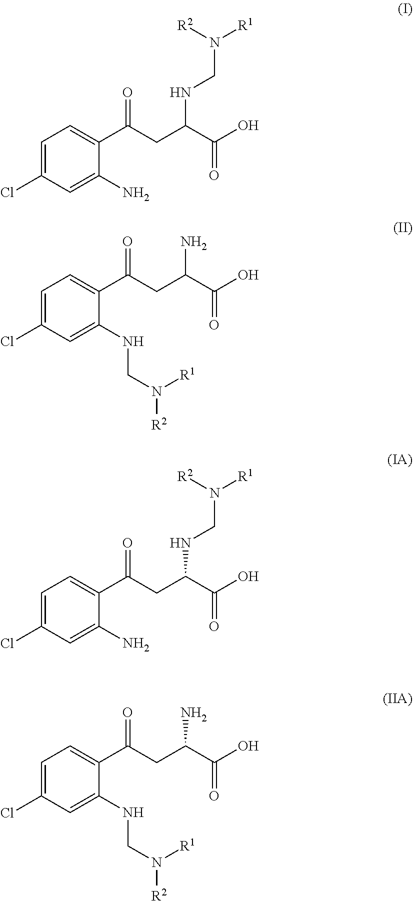

In the structures of formula (I), (IA), (II), and (IIA), R¹ and R² are, independently, optionally substituted C₁₋₆ alkyl, optionally substituted C₃₋₈ cycloalkyl, optionally substituted aryl, optionally substituted heteroaryl, or optionally substituted heterocyclyl. In some embodiments, R¹ and/or R² are, independently, optionally substituted C₁₋₆ alkyl. In other embodiments, R¹ and/or R² are optionally substituted aryl. In further embodiments, R¹ and/or R² are phenyl optionally substituted with one or more of C₁₋₆ alkyl, C₁₋₆ alkoxy, OH, CN, or halogen. In yet other embodiments, R¹ and/or R² are, independently, optionally substituted C₃₋₈ cycloalkyl. In some embodiments, R¹ and/or R² are, independently, optionally substituted heteroaryl. In still other embodiments, R¹ and/or R² are, independently, optionally substituted heterocyclyl. In additional embodiments, R¹ and/or R² are, independently, methyl, ethyl, propyl, butyl, pentyl, hexyl, phenyl, tolyl, cyclobutyl, cyclopentyl, cyclohexyl, cycloheptyl, pyrrolyl, furanyl, piperazinyl, pyridinyl, pyrazinyl, naphthyl, indenyl, benzofuranyl, indolyl, anthryl, or phenanthryl. Alternatively, R¹ and R², together with the atoms to which they are attached, form an optionally substituted 4- to 8-membered heterocyclyl. In some embodiments, R¹ and R² are fused to form a piperazinyl, pyrrolidinyl, azetidinyl, morpholinyl, thiomorpholinyl, dioxothiomorpholinyl, piperidinyl, or piperazinyl.

In other embodiments, compounds having the structure of formula (III) are provided. Enantiomers of the compounds of formula (III) are also contemplated. In certain embodiments, the compound has the structure of formula (IIIA). In other embodiments, the compound has the structure of formula (IIIB). In further embodiments, the compound has the structure of formula (IIIC).

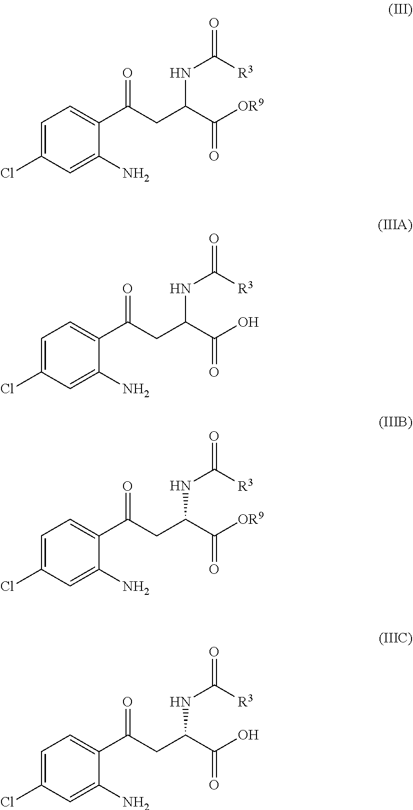

In these structures, R³ is H, optionally substituted C₁₋₆ alkyl, optionally substituted C₁₋₆ alkoxy, optionally substituted arylC₁₋₆ alkyleneoxyl, optionally substituted C₃₋₈ cycloalkyl, optionally substituted aryl, —NH₂, —NHC₁₋₆ alkyl, —N(C₁₋₆ alkyl)₂, optionally substituted heteroaryl, or optionally substituted heterocyclyl and R⁹ is H or optionally substituted C₁₋₆ alkyl. In some embodiments, R³ is C₁₋₆ alkyl. In other embodiments, R³ is C₁₋₆ alkoxy. In yet further embodiments, R³ is optionally substituted arylC₁₋₆ alkyleneoxyl. In still other embodiments, R³ is 9-fluorenylmethyloxyl. In some other embodiments, R³ is optionally substituted C₃₋₈ cycloalkyl. In further embodiments, R³ is optionally substituted aryl. In yet other embodiments, R³ is —NH₂, —NHC₁₋₆ alkyl, or —N(C₁₋₆ alkyl)₂. In still further embodiments, R³ is optionally substituted heteroaryl. In other embodiments, R³ is optionally substituted heterocyclyl. In further embodiments, wherein R⁹ is H. In other embodiments, R⁹ is optionally substituted C₁₋₆ alkyl, for example, methyl, ethyl, propyl, butyl, pentyl, or hexyl.

In some embodiments, compounds having the structure of formula (IV) are provided. Enantiomers of the compounds of formula (IV) are also contemplated. In certain embodiments, the compound has the structure of formula (IVA).

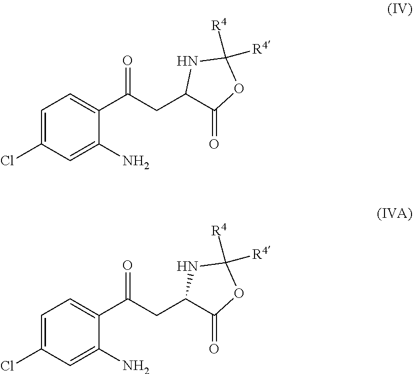

In the structures of formula (IV) and (IVA), R⁴ is H, optionally substituted C₁₋₆ alkyl, optionally substituted C₃₋₈ cycloalkyl, optionally substituted aryl, optionally substituted heteroaryl, or optionally substituted heterocyclyl. R^{4′} is optionally substituted C₁₋₆ alkyl, optionally substituted C₃₋₈ cycloalkyl, optionally substituted aryl, optionally substituted heteroaryl, or optionally substituted heterocyclyl. In some embodiments, R⁴ is H. In further embodiments, R⁴ and/or R^{4′} are optionally substituted C₁₋₆ alkyl. In other embodiments, R⁴ and/or R^{4′} are optionally substituted C₃₋₈ cycloalkyl. In yet further embodiments, R⁴ and/or R^{4′} are optionally substituted aryl. In additional embodiments, R⁴ and/or R^{4′} are optionally substituted heteroaryl. In still other embodiments, R⁴ and/or R^{4′} are optionally substituted heterocyclyl. In further embodiments, R⁴ and/or R^{4′} are H, methyl, ethyl, propyl, butyl, pentyl, hexyl, phenyl, tolyl, cyclobutyl, cyclopentyl, cyclohexyl, cycloheptyl, pyrrolyl, furanyl, piperazinyl, pyridinyl, pyrazinyl, naphthyl, indenyl, benzofuranyl, indolyl, anthryl, or phenanthryl.

In other embodiments, compounds having the structure of formula (V) are provided. Enantiomers of the compounds of formula (V) are also contemplated. In certain embodiments, the compound has the structure of formula (VA). In other embodiments, the compound has the structure of formula (VB). In further embodiments, the compound has the structure of formula (VC).

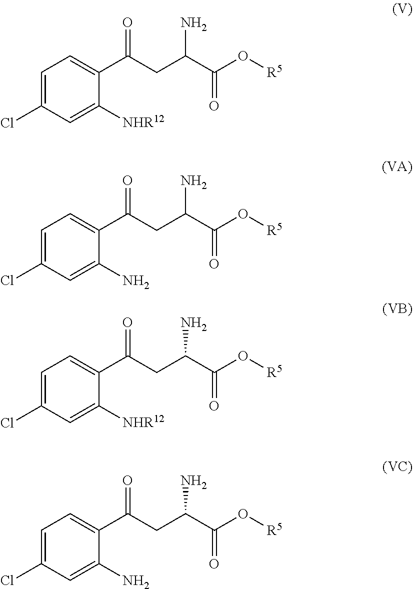

In these structures, R⁵ is optionally substituted C₁₋₁₀ alkyl, optionally substituted aryl, optionally substituted alkylene glycol, —P(O)(OH)₂, —P(O)(OH)(OC₁₋₆alkyl), or —S(O)₂OH and R¹² is H, C(O)C₁₋₆ alkyl, or C(O)OC₁₋₆ alkyl. In some embodiments, R⁵ is optionally substituted C₁₋₁₀ alkyl, for example, methyl, ethyl, propyl, butyl, pentyl, hexyl, heptyl, octyl, nonyl, or decyl. In further embodiments, R⁵ is C₁₋₁₀ alkyl substituted with optionally substituted aryl. In other embodiments, R⁵ is C₁₋₁₀ alkyl substituted with optionally substituted phenyl. In still further embodiments, R⁵ is C₁₋₁₀ alkyl substituted with optionally substituted heterocyclyl. In additional embodiments, R⁵ is C₁₋₁₀ alkyl substituted with optionally substituted tetrahydropyran. In yet further embodiments, R⁵ is C₁₋₁₀ alkyl substituted with tetrahydropyran which is optionally substituted by one, two, three or four C(O)(C₁₋₆ alkyl). In other embodiments, R⁵ is optionally substituted aryl. In further embodiments, R⁵ is —P(O)(OH)₂. In other embodiments, R⁵ is —P(O)(OH)(OC₁₋₆alkyl), for example, —P(O)(OH)(OCH₃), —P(O)(OH)(OCH₂CH₃), —P(O)(OH)(OCH₂CH₂CH₃), or —P(O)(OH)(OCH(CH₃)CH₃). In still other embodiments, R⁵ is —S(O)₂OH. In additional embodiments, R⁵ is optionally substituted alkylene glycol. In additional embodiments, R⁵ is alkylene glycol substituted by C(O)aryl. In further embodiments, R⁵ is alkylene glycol substituted by C(O)phenyl. In other embodiments, R⁵ is OCH₂CH(CH₃)OC(O)(phenyl). In yet further embodiments, R⁵ is —O—CH(CH₃)₂—O—CH(CH₃)₂. In still other embodiments, R⁵ is C₁₋₁₀ alkyl, phenyl, —P(O)(OH)₂, —P(O)(OH)(OC₁₋₆alkyl), or —S(O)₂OH. In some embodiments, R¹² is H. In other embodiments, R¹² is C₁₋₆ alkyl, for example, methyl, ethyl, propyl, butyl, pentyl, or hexyl. In further embodiments, R¹² is C₁₋₆ alkoxy, for example, methoxy, ethoxy, propoxy, butoxy, pentoxy, or hexoxy.

In other embodiments, compounds having the structure of formula (VI) are provided. Enantiomers of the compounds of formula (VI) are also contemplated. In certain embodiments, the compound is the structure of formula (VIA). In further embodiments, the compound is the structure of formula (VIB). In other embodiments, the compound is the structure of formula (VIC).

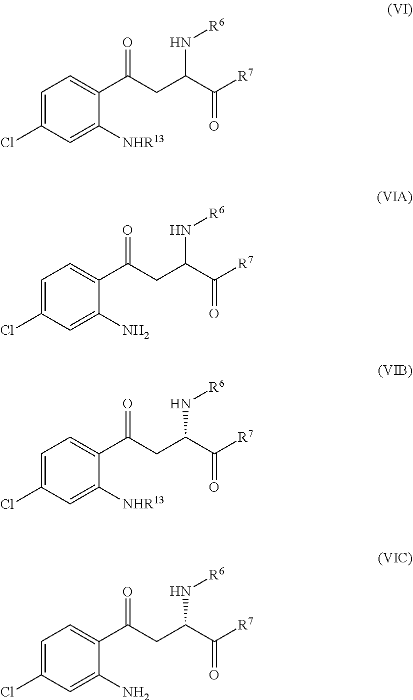

In the structures of formula (VI) and (VIA), R¹³ is H, R⁶ is H, an amino acid moiety, or a peptide moiety and R⁷ is OH, an amino acid moiety, or a peptide moiety, wherein at least one of R⁶ and R⁷ is an amino acid moiety or a peptide moiety comprising at least 2 amino acid moieties. In some embodiments, R⁶ is H. In other embodiments, R¹³ and R⁷ form a bond or CH₂. In further embodiments, R¹³ and R⁷ form a bond. In additional embodiments, R¹³ and R⁷ form a CH₂ group.

In some embodiments, the peptide moiety comprises 2 to about 4 amino acids. In other embodiments, the peptide moiety contains at least two of alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, or valine.

Multimers of the compounds discussed herein are also provided. Multimers are formed by linking two or more of the compounds discussed herein. In certain embodiments, dimers, trimers, and tetramers of the compounds discussed herein are provided. In some embodiments, compounds having the structure of formula (VII) are provided. Enantiomers of the compounds of formula (VII) are also contemplated, wherein one or more monomer is an enantiomer.

monomer 1-linker-monomer 2 (VII)

In the structure of formula (VII), the linker is optionally substituted C₁₋₆ alkyl, optionally substituted C₃₋₈ cycloalkyl, optionally substituted aryl, optionally substituted heteroaryl, or optionally substituted heterocyclyl. Monomer 1 and monomer 2 are independently selected from a moiety of formula (I), (II), or (III) as described above. In certain embodiments, the linker is a glycol moiety. In other embodiments, the linker is —O—(C₁₋₁₀ alkyl-O)ₚ—, where p is 1 to about 10 and each “C₁₋₁₀ alkyl-O” group may differ. In yet other embodiments, the linker is 1,3-propanediol (—O—C₃H₆—O—), 3-(3-hydroxypropoxy)propan-1-ol (—O(CH₂)₃—O—(CH₂)₃O—), or tetraglycol (—O(CH₂CH₂O)₄—).

In further embodiments, compounds having the structure of formula (VIII) are provided. Enantiomers of the compounds of formula (VIII) are also contemplated. In some embodiments, the compound is the structure of formula (VIIIA).

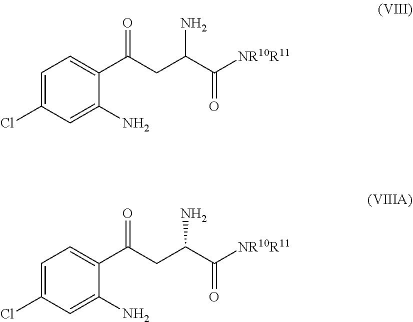

In the structure of formula (VIII), R¹⁰ and R¹¹ are, independently, H, optionally substituted C₁₋₆ alkyl, or SO₂(C₁₋₆ alkyl); or R¹⁰ and R¹¹, together with the atoms to which they are attached, form an optionally substituted heterocyclyl. In some embodiments, R¹⁰ and R¹¹ are, independently, are H. In other embodiments, R¹⁰ and R¹¹ are, independently, optionally substituted C₁₋₆ alkyl, for example, methyl, ethyl, propyl, butyl, pentyl, or hexyl. In further embodiments, R¹⁰ and R¹¹ are, independently, C₁₋₆ alkyl substituted by amino. In yet other embodiments, R¹⁰ and R¹¹ are, independently, C₁₋₆ alkyl substituted by N(CH₃)₂. In still further embodiments, R¹⁰ and R¹¹, are, independently, SO₂(C₁₋₆ alkyl), for example, SO₂(methyl), SO₂(ethyl), SO₂(propyl), SO₂(butyl), SO₂(pentyl), or SO₂(hexyl). In additional embodiments, R¹⁰ and R¹¹ are, independently, C₁₋₆ alkyl substituted by C(O)OH. In other embodiments, R¹⁰ and R¹¹ are, independently, C₁₋₆ alkyl substituted by C(O)C₁₋₆ alkoxy, e.g., C(O)(methoxy), C(O)(ethoxy), C(O)(propoxy), C(O)(butoxy), C(O)(pentoxy), or C(O)(hexoxy). In further embodiments, R¹⁰ and R¹¹ are, independently, C₁₋₆ alkyl, for example, methyl, ethyl, propyl, butyl, pentyl, or hexyl, substituted by optionally substituted aryl. In yet other embodiments, R¹⁰ and R¹¹ are, independently, C₁₋₆ alkyl substituted by optionally substituted phenyl. In still further embodiments, R¹⁰ and R¹¹ are, independently, C₁₋₆ alkyl substituted by OH-substituted phenyl. In some embodiments, R¹⁰ and R¹¹, together with the atoms to which they are attached, form an optionally substituted heterocyclyl. In further embodiments, R¹⁰ and R¹¹, together with the atoms to which they are attached, form an optionally substituted pyrrolidine. In other embodiments, R¹⁰ and R¹¹, together with the atoms to which they are attached, form a pyrrolidone substituted with one or more C₁₋₆ alkyl, for example, methyl, ethyl, propyl, butyl, pentyl, or hexyl.

The above compounds include salts of acidic and basic compounds. In some embodiments, the salts are pharmaceutically acceptable. Pharmaceutically acceptable acid addition salts of compounds described herein include, but are not limited to, salts derived from inorganic acids such as hydrochloric, nitric, phosphoric, sulfuric, hydrobromic, hydroiodic, and phosphoric acids, as well as the salts derived from organic, such as aliphatic mono- and di-carboxylic, phenyl-substituted alkanoic, hydroxy alkanoic, alkanedioic, aromatic, and aliphatic and aromatic sulfonic. Such salts thus include, but are not limited to, sulfate, pyrosulfate, bisulfate, sulfite, bisulfate, nitrate, phosphate, monohydrogenphosphate, dihydrogenphosphate, meta-phosphate, pyrophosphate, chloride, bromide, iodide, acetate, propionate, caprylate, isobutyrate, oxalate, malonate, succinate, suberate, sebacate, fumarate, maleate, mandelate, benzoate, chlorobenzoate, methylbenzoate, dinitrobenzoate, phthalate, benzenesulfonate, toluenesulfonate, phenylacetate, citrate, lactate, maleate, tartrate, and methanesulfonate salts. See, for example, Berge et al., “Pharmaceutical Salts,” J. of Pharmaceutical Science, 1977; 66:1-19.

Acid addition salts may be prepared by contacting a compound described herein with a sufficient amount of the desired acid to produce the salt in the conventional manner. The free base form of a compound described herein may be regenerated by contacting the salt form with a base and isolating the free base in the conventional manner.

Pharmaceutically acceptable base salts of compounds described herein are formed with metals or amines, such as alkali and alkaline earth metal hydroxides, or of organic amines. In certain embodiments, metals used as cations may include, but are not limited to, sodium, potassium, magnesium, and calcium. In other embodiments, amines may include, but are not limited to, N,N′-dibenzylethylenediamine, chloroprocaine, choline, diethanolamine, ethylenediamine (ethane-1,2-diamine), N-methylglucamine, and procaine. See, for example, Berge et al. cited above.

Base addition salts may be prepared by contacting a compound described herein with a sufficient amount of the desired base to produce the salt in the conventional manner. The acid form of the compound described herein may be regenerated by contacting the salt form with an acid and isolating the acid in a conventional manner.

Some compounds described herein may exist as stereoisomers, including enantiomers, diastereomers, and geometric isomers. Some compounds described herein have cycloalkyl groups, which may be substituted at more than one carbon atom, in which case all geometric forms thereof, both cis and trans, and mixtures thereof, are within the scope of the present application. All of these forms, including (R), (S), epimers, diastereomers, cis, trans, syn, anti, (E), (Z), tautomers, and mixtures thereof, are included in the compounds described herein.

Also provided are compositions comprising one or more compound described herein. In certain embodiments, the compositions comprise a compound of one or more of formula (I) to (VIII) and/or a pharmaceutically acceptable salt thereof together with one or more of a pharmaceutically acceptable excipient. Pharmaceutically acceptable excipients are determined in part by the particular composition being administered, as well as by the particular method used to administer the composition. Accordingly, there is a wide variety of suitable formulations of pharmaceutical compositions described herein. See, e.g., Remington: The Science and Practice of Pharmacy, 20th ed., Gennaro et al. Eds., Lippincott Williams and Wilkins, 2000. In some embodiments, such compositions are suitable for pharmaceutical use. Such compositions may be referred to as pharmaceutical compositions. In preparing a pharmaceutical composition from one or more compound described herein, pharmaceutically acceptable excipients can be either solid or liquid. An excipient can be one or more substance which may act as a carrier, diluent, flavoring agent, binder, preservative, tablet disintegrating agent, or an encapsulating material. It should be understood that when the term “excipient” is used, the term can denote any of a carrier, diluent, flavoring agent, binder, preservative, tablet disintegrating agent, and/or encapsulating material. If there is more than one excipient present, the excipients may be of the same general type (i.e., two or more binders) or different types (i.e., a diluent and a preservative).

The pharmaceutical composition may contain two or more compounds described herein. In certain embodiments, two different salt forms of a compound of any one of formula (I) to (VIII) may be used together in the same pharmaceutical composition. In other embodiments, a single composition may contain a mixture of a non-salt and a salt form of the same compound.

The compounds described herein can be formulated as a pharmaceutical composition in any delivery form, such as a syrup, elixir, suspension, powder, granule, tablet, capsule, lozenge, troche, aqueous solution, cream, ointment, lotion, gel, emulsion, etc. Solid form preparations include powders, tablets, pills, capsules, cachets, suppositories, and dispersible granules, among others.

In powders, the excipient may be a finely divided solid in a mixture with a finely divided portion of one or more of the compounds described herein. In tablets, the compounds discussed herein may be mixed with an excipient having the necessary binding properties in suitable proportions and compacted in the shape and size desired. Suitable excipients include magnesium carbonate, magnesium stearate, talc, sugar, lactose, pectin, dextrin, starch, gelatin, tragacanth, methylcellulose, sodium carboxymethylcellulose, low melting wax, cocoa butter, and the like.

For preparing suppositories, a low melting wax, such as a mixture of fatty acid glycerides or cocoa butter, may be melted and one or more compound discussed herein dispersed homogeneously therein. The molten homogeneous mixture may then be poured into convenient sized molds, allowed to cool, and thereby to solidify.

Liquid form preparations include solutions, suspensions, and emulsions. Formulations suitable for parenteral administration include aqueous and non-aqueous, isotonic sterile injection solutions, which can contain antioxidants, buffers, bacteriostats, and solutes that render the formulation isotonic with the blood of the intended recipient, and aqueous and nonaqueous sterile suspensions that can include suspending agents, solubilizers, thickening agents, stabilizers, and preservatives. The formulations of compounds discussed herein may be presented in unit-dose or multi-dose sealed containers, such as ampoules and vials. Injection solutions and suspensions can be prepared from sterile powders, granules, and tablets of the kind previously described.

One or more compound described herein, alone or in combination with other suitable components, can be made into aerosol formulations, e.g., they can be “nebulized,” to be administered via inhalation. Aerosol formulations can be placed into pressurized acceptable propellants, such as dichlorodifluoromethane, propane, nitrogen, and the like.

The compositions may also contain, in addition to a compound of any one of formula (I) to (VIII) or a pharmaceutically acceptable salt thereof and a pharmaceutically acceptable excipient, an additional therapeutic compound, such as a compound useful in the treatment of depression. In certain embodiments, the additional therapeutic compound is L-DOPA.

The pharmaceutical composition may contain a therapeutically effective amount of a compound of any one of formula (I) to (VIII) and/or a pharmaceutically acceptable salt thereof. In certain embodiments, the compositions contain an amount of a compound of any one of formula (I) to (VIII) and/or a pharmaceutically acceptable salt thereof which is effective to treat an NMDA related disorder or condition. The amount of the compounds discussed herein in the pharmaceutical composition may be varied or adjusted according to the particular application and the desired size of the dosage form.

The dose of one or more compound discussed herein administered to a subject is sufficient to induce a beneficial therapeutic response in the subject over time. The beneficial dose can vary from subject to subject depending upon the subject's condition, body weight, surface area, and side effect susceptibility, among others. Administration can be accomplished via single or divided doses.

As discussed above, the compounds described herein modulate the NMDA receptor. In some embodiments, the compounds described herein are NMDA antagonists. In further embodiments, the compounds described herein are vesicular glutamate reuptake antagonists. In other embodiments, the compounds discussed herein will cause a decrease in symptoms or disease indicia associated with an NMDA related disorder.

Further provided are methods of treating conditions requiring modulation of the NMDA receptor. In certain embodiments, methods for treating conditions requiring modulation of the NMDA receptor using compounds of any one of formula (I) to (VIII) as defined herein and/or a pharmaceutically acceptable salt thereof are provided. In other embodiments, a compound of any one of formula (I) to (VIII) as defined herein and/or a pharmaceutically acceptable salt thereof is provided for use in the preparation of a medicament for treating a NMDA-related disorder or condition in a subject.

Accordingly, the compounds discussed herein may be used in the treatment of a variety of conditions, including those modulated by the NMDA receptor. In some embodiments, the compounds discussed herein are useful in methods for treating a neurodegenerative disorder. One skilled in the art would be able to determine the type of neurodegenerative disorder responsive to the compounds discussed herein. In one embodiment, the neurodegenerative disorder is an age-related cognitive disorder or a perinatal brain disorder. In another embodiment, the neurodegenerative disorder is Alzheimer's disease, vascular dementia, Parkinson's disease, or traumatic brain injury.

In other embodiments, the compounds discussed herein are useful in methods for enhancing learning, memory, or cognition in a patient. In further embodiments, the compounds discussed herein are useful in methods of treating conditions caused by neurological dysfunction. In certain embodiments, the compounds discussed herein are useful in methods of treating depression. In still other embodiments, the compounds discussed herein are useful in methods of treating major depressive disorder. In one embodiment, the major depressive disorder is biopolar disorder. In yet further embodiments, the compounds discussed herein are useful in methods of treating hyperalgesia. In some embodiments, the compounds discussed herein may be used in methods for reducing an L-DOPA associated dyskinesia.

The NMDA related disorder or condition can be treated prophylactically, acutely, or chronically using compounds described herein, depending on the nature of the disorder or condition.

The compounds described herein may be administered in combination with one or more additional active agents. The additional active agent may be administered to the patient prior to, concurrently with, or subsequent to the compounds discussed herein. Accordingly, the additional active agent may be in a combination pharmaceutical product together with one or more compound discussed herein. In certain embodiments, the other active agents are effective in treating the NMDA related disorder or condition. In other embodiments, the other active agents include, without limitation, L-DOPA.

The compounds described herein may be prepared and administered in a wide variety of dosage forms. Thus, the compounds may be administered by injection, (intravenously, intramuscularly, intracutaneously, subcutaneously, intraduodenally, intraperitoneally, intrathecally, intravesically), inhalation (intranasally), transdermally, orally, rectally, bucally, topically, or by insufflation.

Determination of the proper dosage for a particular situation is within the skill of the practitioner. Generally, treatment is initiated with smaller dosages which are less than the optimum dose of the compound. Thereafter, the dosage is increased by small increments until the optimum effect under the circumstances is reached. For convenience, the total daily dosage may be divided and administered in portions during the day, if desired. In certain embodiments, a dose is about 1 mg to about 1,000 mg per day, such as about 5 mg to about 500 mg per day. In other embodiments, the dose is about 10 mg to about 300 mg per day, such as about 25 mg to about 250 mg per day.

### EXAMPLES

### Example A: General Synthesis of 4-Chlorokynurenine Esters

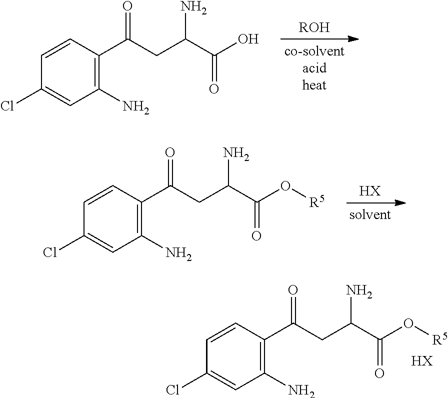

Preparation of esters of 4-chlorokynurenine uses a substituted alcohol, neat, or with a high boiling co-solvent, such as toluene, with a mineral acid, such as hydrochloric acid (HCl) (3 to 4 equivalents) at elevated temperature, 80° C. to 120° C., for 1 to 48 hours. The solvent and excess alcohol evaporates under reduced pressure. Purification utilizes chromatography, normal or reverse phase, or, precipitation in the form of a salt using a mineral or organic acid, such as hydrogen chloride, hydrogen bromide, sulfuric acid, methanesulfonic acid, camphorsulfonic acid (CSA), p-toluenesulfonic acid (p-TSA), etc., from an organic solvent, such as ether, tetrahydrofuran (THF), p-dioxane, toluene, ethyl acetate (EtOAc), or a mixture thereof.

### Example 1: Ethyl 2-amino-4-(2-amino-4-chlorophenyl)-4-oxo-butanoate

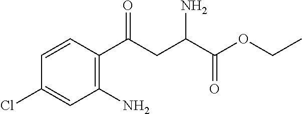 [SMILES: CCOC(=O)C(N)CC(=O)c1ccc(Cl)cc1N]

A reaction tube with a stir bar was charged with 2-amino-4-(2-amino-4-chloro-phenyl)-4-oxo-butanoic acid (0.0750 g, 0.309 mmol), ethanol (2 mL) and hydrogen chloride (4.0 M in 1,4-dioxane) (0.325 g, 0.309 mL, 1.24 mmol). The tube was sealed and heated at 90° C. overnight. The volatiles were evaporated. The residue was purified via reverse phase chromatography using 10% to 50% acetonitrile:water (w/0.1% trifluoroacetic acid (TFA) as modifier) solvent gradient. The desired fractions were combined, frozen and lyophilized. The resulting lyophilate TFA salt was dissolved in acetonitrile (ACN) (2 mL) and methanesulfonic acid (50 μL) was added with stirring at room temperature. A precipitate was observed after several minutes. The solid was filtered, rinsed with acetonitrile and dried by suction. Ethyl 2-amino-4-(2-amino-4-chlorophenyl)-4-oxo-butanoate (0.0669 g, 0.145 mmol, 46.8% Yield), as the bis-mesylate salt, was recovered as a white solid. ¹H NMR (400 MHz, DMSO-d₆) δ 8.47-8.18 (m, 3H), 7.76 (d, J=8.8 Hz, 1H), 7.69-7.11 (m, 1H), 6.88 (d, J=2.3 Hz, 1H), 6.60 (dd, J=8.7, 2.1 Hz, 1H), 4.43 (br. s., 1H), 4.24-4.14 (m, 2H), 3.69-3.52 (m, 2H), 2.31 (s, 6H), 1.18 (t, J=7.0 Hz, 3H). MS=270.93, 272.91 (MH)⁺ (chlorine motif).

### Example 2: Methyl 2-amino-4-(2-amino-4-chlorophenyl)-4-oxo-butanoate

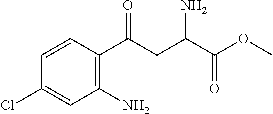 [SMILES: COC(=O)C(N)CC(=O)c1ccc(Cl)cc1N]

Methyl 2-amino-4-(2-amino-4-chlorophenyl)-4-oxo-butanoate was prepared from 2-amino-4-(2-amino-4-chlorophenyl)-4-oxo-butanoic acid in an analogous manner to Example 1. The product was isolated as white solid (0.0534 g, 0.119 mmol, 38.5% Yield) as the bis-mesylate salt. ¹H NMR (400 MHz, DMSO-d₆) δ 8.47-8.19 (m, 3H), 7.75 (d, J=8.8 Hz, 1H), 7.70-7.02 (m, 1H), 6.88 (d, J=2.3 Hz, 1H), 6.60 (dd, J=8.8, 2.3 Hz, 1H), 4.48-4.43 (m, 1H), 3.73 (s, 3H), 3.70-3.54 (m, 2H), 2.33 (s, 6H). MS=256.92, 258.88 (MH)⁺ (chlorine motif).

### Example 3: Isopropyl 2-amino-4-(2-amino-4-chlorophenyl)-4-oxo-butanoate

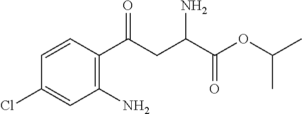 [SMILES: CC(C)OC(=O)C(N)CC(=O)c1ccc(Cl)cc1N]

Isopropyl 2-amino-4-(2-amino-4-chlorophenyl)-4-oxo-butanoate was prepared from 2-amino-4-(2-amino-4-chlorophenyl)-4-oxo-butanoic acid in an analogous manner to Example 1. The product was isolated as an off-white solid (0.0335 g, 0.0702 mmol, 22.7% Yield) as the bis-mesylate salt. ¹H NMR (400 MHz, DMSO-d₆) δ 8.44-8.11 (m, 3H), 7.76 (d, J=8.8 Hz, 1H), 7.67-7.11 (m, 1H), 6.88 (d, J=2.0 Hz, 1H), 6.60 (dd, J=8.7, 2.1 Hz, 1H), 5.04-4.93 (m, 1H), 4.43-4.34 (m, 1H), 3.68-3.47 (m, 2H), 2.36-2.26 (m, 6H), 1.22 (d, J=6.3 Hz, 3H), 1.14 (d, J=6.0 Hz, 3H). MS=284.95, 286.92 (MH)⁺ (chlorine motif).

### Example 4: Propyl 2-amino-4-(2-amino-4-chlorophenyl)-4-oxo-butanoate

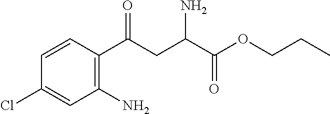 [SMILES: CCCOC(=O)C(N)CC(=O)c1ccc(Cl)cc1N]

Propyl 2-amino-4-(2-amino-4-chlorophenyl)-4-oxo-butanoate was prepared from 2-amino-4-(2-amino-4-chlorophenyl)-4-oxo-butanoic acid in an analogous manner to Example 1. The product was isolated as an off-white solid (0.0468 g, 0.0981 mmol, 31.7% Yield) as the bis-mesylate salt. ¹H NMR (400 MHz, DMSO-d₆) δ 8.30 (d, J=3.5 Hz, 3H), 7.76 (d, J=8.8 Hz, 1H), 7.38 (br. s., 1H), 6.88 (d, J=2.0 Hz, 1H), 6.60 (dd, J=8.8, 2.0 Hz, 1H), 4.48-4.40 (m, 1H), 4.16-4.04 (m, 2H), 3.72-3.52 (m, 2H), 2.32 (s, 6H), 1.57 (sxt, J=7.1 Hz, 2H), 0.83 (t, J=7.4 Hz, 3H). MS=284.94, 286.92 (MH)⁺ (chlorine motif).

### Example 5: 4-(2-amino-4-chlorophenyl)-2-(tert-butoxycarbonylamino)-4-oxo-butanoic acid

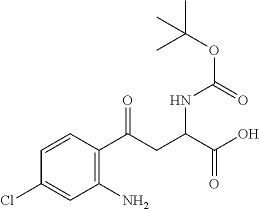 [SMILES: CC(C)(C)OC(=O)NC(CC(=O)c1ccc(Cl)cc1N)C(=O)O]

To a stirred suspension of 2-amino-4-(2-amino-4-chlorophenyl)-4-oxo-butanoic acid (0.2000 g, 0.8242 mmol) and triethylamine (TEA) (0.1264 g, 0.174 mL, 1.236 mmol) in water (1 mL) and 1,4-dioxane (1 mL) was added tert-butoxycarbonyl tert-butyl carbonate (0.1979 g, 0.9066 mmol). The mixture was stirred for 3 hours until a clear yellow solution resulted. The reaction mixture was diluted with water (10 mL) and extracted with ether (3×10 mL). The aqueous layer was acidified with 1N HCl (1 mL) then extracted with EtOAc (3×10 mL). The combined EtOAc layers were dried over sodium sulfate (Na₂SO₄), filtered and the filtrate was evaporated to a yellow foam. The foam was dissolved in dichloromethane (DCM) 1 mL) and hexane (2 mL) was added to precipitate the solid. The volatiles were evaporated and the solid was subjected to high vacuum for 2 hours. 4-(2-Amino-4-chlorophenyl)-2-(tert-butoxy-carbonylamino)-4-oxo-butanoic acid (0.256 g, 0.747 mmol, 90.6% Yield) was recovered as a yellow solid. ¹H NMR (400 MHz, DMSO-d₆) δ 12.56 (br. s, 1H), 7.73 (d, J=8.8 Hz, 1H), 7.38 (br. s., 2H), 6.96 (d, J=8.0 Hz, 1H), 6.83 (d, J=2.0 Hz, 1H), 6.55 (dd, J=8.7, 2.1 Hz, 1H), 4.50-4.40 (m, 1H), 3.40-3.20 (m, 2H), 1.36 (s, 9H). MS=364.90 (M+Na)⁺.

### Example 6: 2-acetamido-4-(2-amino-4-chloro-phenyl)-4-oxo-butanoic acid

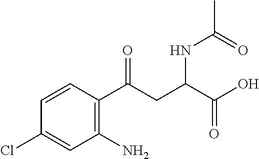 [SMILES: CC(=O)NC(CC(=O)c1ccc(Cl)cc1N)C(=O)O]

To a suspension of 2-amino-4-(2-amino-4-chlorophenyl)-4-oxo-butanoic acid (0.0500 g, 0.206 mmol) and TEA (0.0316 g, 0.0435 mL, 0.309 mmol) in water (0.5 mL) and 1,4-dioxane (0.5 mL) was added EtOAc (0.0231 g, 0.227 mmol). The mixture was stirred at room temperature for 2 hours until a clear yellow solution resulted. The mixture was acidified with 1N HCl (1 mL) and the volatiles were evaporated. The residue was purified via reverse phase chromatography using 10% to 50% ACN:water (w/0.1% TFA as modifier) solvent gradient. The desired fraction was frozen and lyophilized. The recovered pale yellow lyophilate was 2-acetamido-4-(2-amino-4-chlorophenyl)-4-oxo-butanoic acid (0.0452 g, 0.113 mmol, 55.0% Yield) as the trifluoroacetic acid salt. ¹H NMR (400 MHz, DMSO-d₆) δ 8.11 (d, J=7.8 Hz, 1H), 7.76 (d, J=8.8 Hz, 1H), 7.70-6.91 (m, 1H), 6.83 (d, J=2.0 Hz, 1H), 6.55 (dd, J=8.8, 2.0 Hz, 1H), 4.73-4.63 (m, 1H), 3.33 (d, J=6.0 Hz, 2H), 1.81 (s, 3H). MS=284.91, 286.89 (MH)⁺ (chlorine motif).

### Example 7: Synthesis of 4-Chlorokynurenine Phosphate Ester Prodrugs

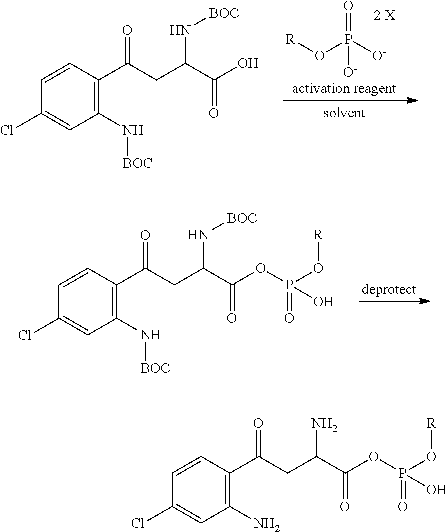

The preparation of phosphate esters of 4-chlorokynurenine uses Nα,N′-bis-BOC-4-chlorokynurenine, an activation reagent, such as DCC, in a solvent, such as DCM or water, utilizing a substituted bis-tetraalkonium phosphate ester. The solvent evaporates under reduced pressure. Purification of the residue utilizes chromatography, normal or reverse phase. An acid, such as TFA, in a solvent, such as DCM, deprotects the intermediate. The solvent and acid evaporates under reduced pressure and purification requires chromatography, reverse phase or ion.

### Example 8: Synthesis of 4-Chlorokynurenine Sulfate Ester Prodrug

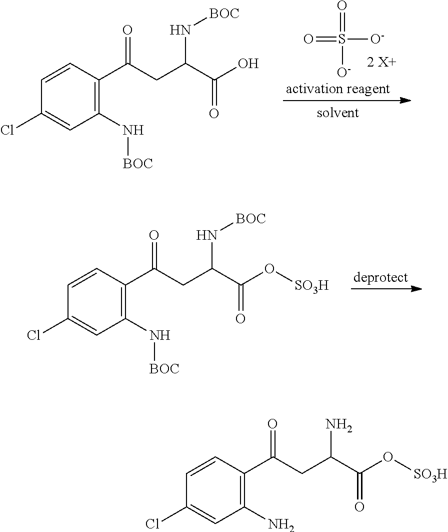 [SMILES: CC(C)(C)OC(=O)Nc1cc(Cl)ccc1C(=O)CC(NC(=O)OC(C)(C)C)C(=O)O ; O=S(=O)([O-])[O-] ; CC(C)(C)OC(=O)Nc1cc(Cl)ccc1C(=O)CC(NC(=O)OC(C)(C)C)C(=O)OS(=O)(=O)O ; Nc1cc(Cl)ccc1C(=O)CC(N)C(=O)OS(=O)(=O)O]

Preparation of sulfate esters of 4-chlorokynurenine uses Nα,N′-bis-BOC-4-chlorokynurenine, an activation reagent, such as dicyclohexylcarbodiimide (DCC), in a solvent, such as DCM or water, utilizing a substituted bis-tetraalkonium sulfate ester. The solvent evaporates under reduced pressure. Purification of the residue utilizes chromatography, reverse phase. An acid, such as TFA, in a solvent, such as DCM, deprotects the intermediate. The solvent and acid evaporates under reduced pressure and purification requires chromatography, reverse phase or ion.

### Example 9: Synthesis of Nα-Substituted 4-Chlorokynurenine Prodrugs

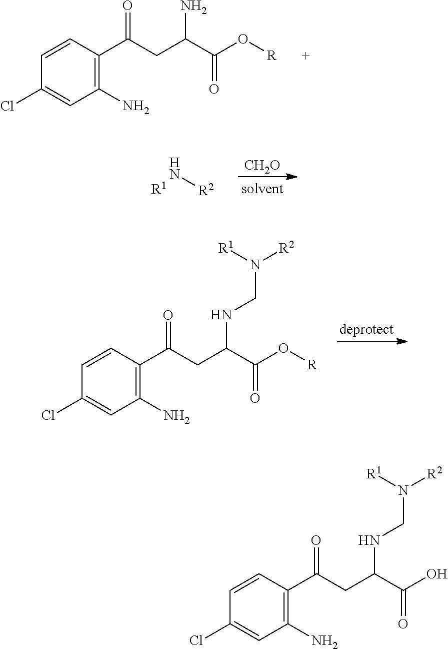

Preparation of Nα-substituted 4-chlorokynurenines uses a substituted ester of 4-chlorokynurenine, such as the ethyl ester, with a substituted amine and aqueous formaldehyde, or equivalent, in a solvent, such as methanol or ethanol, at room temperature, or elevated temperature, such as 26° C. to 100° C. The solvent evaporates under reduced pressure and purification utilizes chromatography, normal or reverse phase. The ester dissolves in an alcoholic solvent mixture, such as methanol or ethanol, and stirs with an aqueous solution of a hydroxide base, such as lithium, sodium or potassium hydroxide at room temperature, or elevated temperature, such as 26° C. to 100° C., for 1 to 48 hours. An acid, such as acetic acid neutralizes the mixture. Solvent and acid evaporates under reduced pressure and purification utilizes chromatography, normal or reverse phase.

### Example 10: Synthesis of N′-Substituted 4-Chlorokynurenine Prodrugs

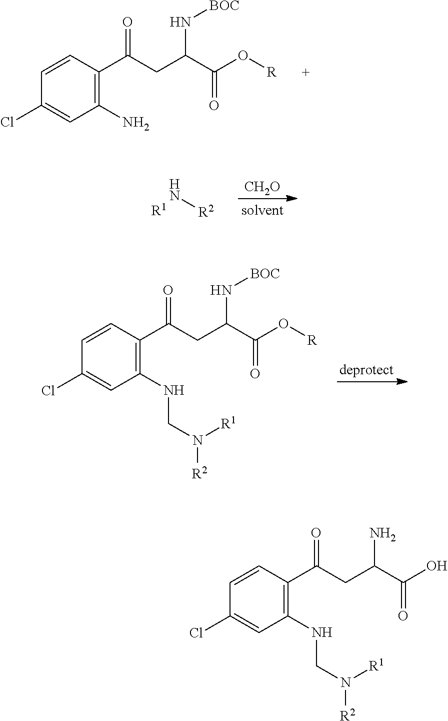

Preparation of N′-substituted 4-chlorokynurenines uses a Nα-protected substituted ester of 4-chlorokynurenine, such as Nα-BOC-4-chlorokynurenine ethyl ester, a substituted amine and aqueous formaldehyde, or equivalent, in a solvent, such as methanol or ethanol, at room temperature, or elevated temperature, such as 26 to 100° C. The solvent evaporates under reduced pressure and purification utilizes chromatography, normal or reverse phase. An acid, such as TFA, removes the BOC group. The ester dissolves in an alcoholic solvent mixture, such as methanol or ethanol, and stirs with an aqueous solution of a hydroxide base, such as lithium, sodium or potassium hydroxide at room temperature, or elevated temperature, such as 26 to 100° C., for 1 to 48 hours. An acid, such as acetic acid, neutralizes the mixture. Solvent and acid evaporates under reduced pressure and purification utilizes chromatography, normal or reverse phase.

### Example 11: Synthesis of Cyclic Amino Acid Prodrugs of 4-Chlorokynurenine

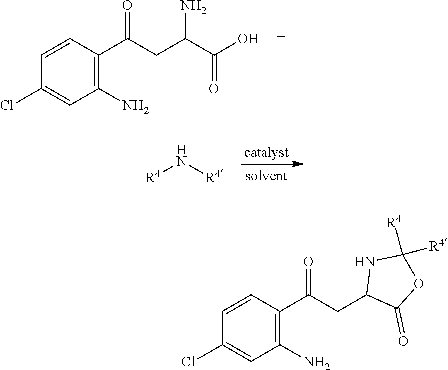

Preparation of cyclic amino acid 4-chlorokynurenines uses a substituted aldehyde or ketone or synthetic equivalent, such as a hydrate, acetal or hemiacetal, with a catalyst, such as p-TSA or CSA, and a solvent, such as acetonitrile, acetone, methanol or ethanol. The mixture stirs at room temperature, or an elevated temperature, from 26 to 130° C., for 1 to 48 hours. The solvent evaporates under reduced pressure and purification utilizes chromatography, normal or reverse phase.

### Example 12: Synthesis of 4-Chlorokynurenine Amino Acid Prodrugs

A. Amino Acid Prodrugs Bound Through a Carbon Atom

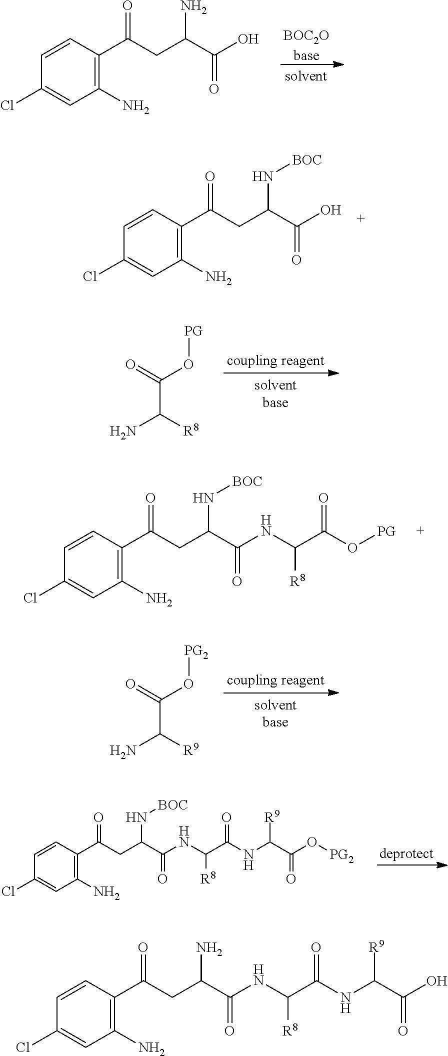

Preparation of amino acid derivatives of 4-chlorokynurenine uses protected Nα-BOC-4-chlorokynurenine, a peptide coupling reagent, such as Woodward's reagent K or isobutylchloroformate, in a solvent, such as ACN or dimethylformamide (DMF), with an amine base, such as trimethylamine, TEA or N-methylmorpholine, and a protected amino acid ester. The mixture stirs at a temperature, such as −15° C. to room temperature, for 1 to 48 hours. The reaction utilizes solution phase or solid support conditions. Successive coupling of other protected amino acid esters react in a similar manner. Finally, acidic deprotection conditions, such as HCl, hydrobromic acid or TFA, with a cation scavenger, such as anisole, removes the protecting groups. Purification utilizes chromatography, reverse phase or ion.

B. Amino Acid Prodrugs Bound Through a Nitrogen Atom

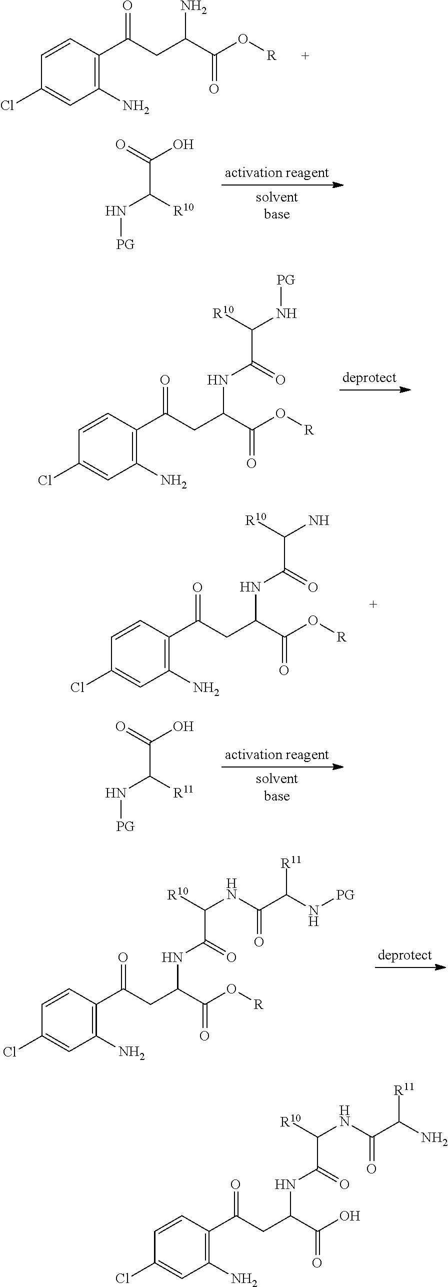

Preparation of amino acid derivatives of 4-chlorokynurenine uses a protected 4-chlorokynurenine ester and an activated N-protected amino acid, such as N-FMOC glycine-OBt or N—BOC glycine-OSu. Preparation of the activated amino acid esters uses an activation reagent, such as diisopropyl carbodiimide (DIC) or isobutylchloroformate, and a leaving group, such as HOSu, HOBt or p-nitrophenol. The mixture stirs in a solvent, such as ACN, DMF or N-methylpyrrolidinone (NMP), water or acetone, or mixture thereof, with a base, such as trimethylamine, TEA, N-methylmorpholine or sodium bicarbonate (NaHCO₃), at a temperature, such as from about −15° C. to about room temperature for 1 to 48 hours. The reaction utilizes solution phase or solid support conditions. Cleavage conditions with an acid, such as TFA, or with a base, such as piperidine, deprotects the amino acid intermediate and allows the successive coupling of other activated N-protected amino acids. Finally, deprotection conditions, basic or acidic, such as piperidine or HCl, hydrobromic acid or TFA, with a cation scavenger, such as anisole, removes the protecting groups. Purification utilizes chromatography, reverse phase or ion.

### Example 13: Synthesis of Carbamate Prodrugs

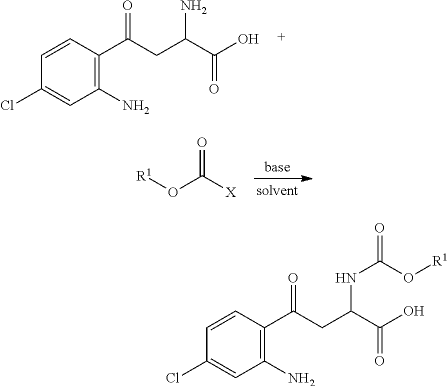

Preparation of Nα-carbamate derivatives of 4-chlorokynurenine uses 4-chlorokynurenine, or salt thereof, such as hydrochloride, hydrobromide or sulfate, a substituted carbamoylating reagent, like an anhydride, such as di-tert-butyl dicarbonate or diethyl dicarbonate, or an activated reagent, such as Boc-ON, Boc-OSu, FMOC-OSu, or a chloroformate, such as ethyl chloroformate or phenyl chloroformate. The mixture stirs with a base, such as trimethylamine, TEA or NaHCO₃, in a solvent such as water, acetone, THF, p-dioxane, or mixture thereof, at a temperature, such as from about −5° C. to about room temperature for about 1 to about 48 hours. Purification utilizes chromatography, normal or reverse phase.

### Example 14: Synthesis of Acyl Prodrugs

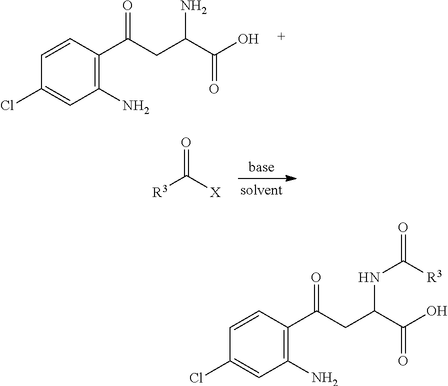

Preparation of Nα-acyl derivatives of 4-chlorokynurenine uses 4-chlorokynurenine, or salt thereof, such as hydrochloride, hydrobromide or sulfate, a substituted acylation reagent, like an anhydride, such as acetic anhydride or benzoic anhydride, or activated acylation reagent, such as an acylimidazole, propionyl-OSu, benzoyl-OSu, or an acid chloride, such as acetyl chloride or benzoyl chloride. The mixture stirs with a base, such as trimethylamine, TEA or NaHCO₃, in a solvent such as water, acetone, THF, p-dioxane, or a mixture thereof, at a temperature, such as −5° C. to room temperature. Purification utilizes chromatography, reverse phase or ion.

### Example 15: 3-[2-amino-4-(2-amino-4-chlorophenyl)-4-oxo-butanoyl]oxypropyl 2-amino-4-(2-amino-4-chloro-phenyl)-4-oxo-butanoate

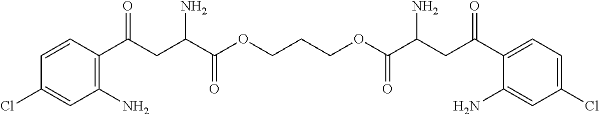 [SMILES: Nc1cc(Cl)ccc1C(=O)CC(N)C(=O)OCCCOC(=O)C(N)CC(=O)c1ccc(Cl)cc1N]

To a suspension of 4-(2-amino-4-chlorophenyl)-2-(tert-butoxycarbonylamino)-4-oxo-butanoic acid (0.1016 g, 0.2964 mmol) and 1,2-dichloroethane (1 mL) was added 1,1′-carbonyldiimidazole (CDI) (0.0587 g, 0.362 mmol) and the resulting suspension was stirred at room temperature for 20 minutes. To the solution was added 1,3-propanediol (0.0121 g, 0.159 mmol) and the mixture was stirred at room temperature overnight. A mixture of the desired material and the hydroxypropyl mono ester were observed. The reaction mixture was loaded onto silica gel (5 g) and purified via chromatography using silica gel (12 g) and 0% to 100% ETOAc:hexane solvent gradient to separate the dimer followed by solvent switch using 0% to 5% methanol:DCM to elute the hydroxypropyl mono ester.

3-[4-(2-amino-4-chlorophenyl)-2-(tert-butoxycarbonylamino)-4-oxo-butanoyl]-oxypropyl 4-(2-amino-4-chlorophenyl)-2-(tert-butoxycarbonylamino)-4-oxo-butanoate was dissolved in DCM (1 mL) and TFA (0.5 mL) was added. The mixture was stirred for 20 minutes at room temperature. The volatiles were evaporated and the residue was purified via reverse phase chromatography using 15% to 60% ACN:water (w/0.1% TFA as modifier). The desired fractions were combined, frozen and lyophilized. The lyophilate was hygroscopic. The residue was dissolved in ACN (1 mL) and p-toluenesulfonic acid monohydrate (40.0 mg) was added, then stirred at room temperature. The ACN supernatant was decanted and the resin was rinsed with dry ACN several times. The resin was dissolved in methanol, transferred to a tared vial, evaporated and placed under high vacuum overnight to yield a tan foam consistent for 3-[2-amino-4-(2-amino-4-chlorophenyl)-4-oxo-butanoyl]oxypropyl 2-amino-4-(2-amino-4-chloro-phenyl)-4-oxo-butanoate as the tris-p-toluenesulfonic acid salt (0.0443 mg, 14% yield). ¹H NMR (400 MHz, DMSO-d₆) δ 8.28 (d, J=4.8 Hz, 6H), 7.73 (dd, J=8.9, 0.9 Hz, 2H), 7.50-7.45 (m, 6H), 7.40 (br. s., 3H), 7.14-7.08 (m, 6H), 6.87 (dd, J=6.1, 2.1 Hz, 2H), 6.59 (dt, J=8.8, 2.0 Hz, 2H), 4.43 (d, J=4.0 Hz, 2H), 4.27-4.12 (m, 4H), 3.67-3.53 (m, 4H), 2.29 (s, 9H), 1.96-1.84 (m, 2H). MS=525.03, 527.03, 529.03 (MH)+(di-chloro motif).

### Example 16: 2-[2-[2-[2-[2-amino-4-(2-amino-4-chlorophenyl)-4-oxo-butanoyl]oxyethoxy]ethoxy]ethoxy]ethyl 2-amino-4-(2-amino-4-chlorophenyl)-4-oxo-butanoate

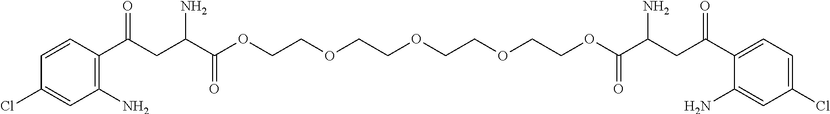 [SMILES: Nc1cc(Cl)ccc1C(=O)CC(N)C(=O)OCCOCCOCCOCCOC(=O)C(N)CC(=O)c1ccc(Cl)cc1N]

2-[2-[2-[2-[2-amino-4-(2-amino-4-chlorophenyl)-4-oxo-butanoyl]oxyethoxy]-ethoxy]ethoxy]ethyl 2-amino-4-(2-amino-4-chlorophenyl)-4-oxo-butanoate was prepared from 4-(2-amino-4-chlorophenyl)-2-(tert-butoxycarbonylamino)-4-oxo-butanoic acid (0.1038 g, 0.3028 mmol) and tetraethylene glycol (0.0294 g, 0.0261 mL, 0.151 mmol) in a manner analogous to Example 15. Product was isolated a pale yellow lyophilate (0.0254 g, 8% yield) as the tetra-trifluoroacetic acid salt. ¹H NMR (400 MHz, DMSO-d₆) δ 8.38 (br. s., 6H), 7.74 (d, J=8.8 Hz, 2H), 7.69-7.07 (m, 3H), 6.87 (d, J=2.3 Hz, 2H), 6.59 (dd, J=8.7, 2.1 Hz, 2H), 4.45 (br. s., 2H), 4.34-4.14 (m, 4H), 3.70-3.57 (m, 8H), 3.41-3.29 (m, 8H). MS=643.15, 645.15, 647.14 (MH)+ (di-chloro motif).

### Example 17: Butyl 2-amino-4-(2-amino-4-chlorophenyl)-4-oxo-butanoate

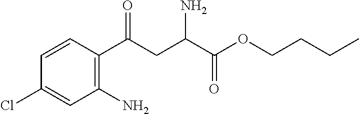 [SMILES: CCCCOC(=O)C(N)CC(=O)c1ccc(Cl)cc1N]

A 1 dram vial with screw cap and stir bar was charged with 4-(2-amino-4-chlorophenyl)-2-(tert-butoxycarbonylamino)-4-oxo-butanoic acid (0.0660 g, 0.193 mmol) and 1,2-dichloroethane (1 mL) and was stirred at room temperature. To the yellow suspension was added CDI (0.0375 g, 0.231 mmol) and stirred at room temperature for 1 hour yielding a yellow solution. 1-Butanol (0.0285 g, 0.0353 mL, 0.385 mmol) was added and the mixture was stirred at room temperature overnight. The volatiles were evaporated onto silica gel (5 g) and purified via chromatography using silica gel column (12 g) and 0% to 80% ETOAc:hexane solvent gradient. The desired fractions were combined and evaporated. The residue was consistent for desired butyl 4-(2-amino-4-chlorophenyl)-2-(tert-butoxycarbonylamino)-4-oxo-butanoate (0.0354 g, 0.0887 mmol, 46.1% Yield) and used without further purification.

Butyl 4-(2-amino-4-chlorophenyl)-2-(tert-butoxycarbonylamino)-4-oxo-butanoate (0.0354 g, 0.0887 mmol) was dissolved in DCM (0.5 mL) and TFA (0.7 g, 0.5 mL, 6 mmol) was added and was stirred at room temperature for 20 minutes. The volatiles were evaporated and subjected to high vacuum for 30 minutes. The residue was dissolved in ACN (1 mL) and methanesulfonic acid (0.0191 g, 0.0130 mL, 0.198 mmol) was added. A suspension resulted within 1-2 minutes. The mixture was stirred for 15 minutes then filtered, rinsed with ACN and partially dried by suction. The solid was subjected to high vacuum for 3 hours. The recovered off-white solid was consistent for butyl 2-amino-4-(2-amino-4-chlorophenyl)-4-oxo-butanoate as the bis-mesylate salt (0.0370 g, 0.0754 mmol, 39.1% Yield). 1H NMR (400 MHz, DMSO-d6) δ 8.39-8.21 (m, 3H), 7.76 (d, J=8.8 Hz, 1H), 7.41 (br. s., 2H), 6.88 (d, J=2.3 Hz, 1H), 6.60 (dd, J=8.7, 2.1 Hz, 1H), 4.49-4.38 (m, 1H), 4.24-4.04 (m, 2H), 3.72-3.49 (m, 2H), 2.32 (s, 6H), 1.60-1.46 (m, 2H), 1.33-1.19 (m, 2H), 0.82 (t, J=7.3 Hz, 3H); LC/MS=298.94, 300.92 (MH)+; chlorine motif.

### Example 18: [(1R)-2-[2-amino-4-(2-amino-4-chlorophenyl)-4-oxo-butanoyl]oxy-1-methyl-ethyl] benzoate

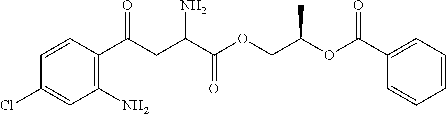 [SMILES: C[C@H](COC(=O)C(N)CC(=O)c1ccc(Cl)cc1N)OC(=O)c1ccccc1]

To a stirred solution of (2R)-propane-1,2-diol (1.008 g, 13.25 mmol) and imidazole (0.8922 g, 0.866 mL, 13.11 mmol) in DCM (10 mL) at 0° C. was added tert-butyldimethyl-chlorosilane (2.008 g, 12.92 mmol). The mixture was stirred for 1 hour cold. The resulting suspension was filtered, rinsed with DCM (5 mL) and the filtrate was evaporated. The resulting clear oil was consistent for desired (2R)-1-[tert-butyl(dimethyl)silyl]oxypropan-2-ol (2.52 g, 13.2 mmol, 99.9% Yield). 1H NMR (400 MHz, DCCl₃) δ 3.79-3.69 (m, 1H), 3.52 (dd, J=9.9, 3.4 Hz, 1H), 3.27 (dd, J=9.9, 7.9 Hz, 1H), 2.14 (br. s, 1H), 1.04 (d, J=6.5 Hz, 3H), 0.83 (s, 9H), 0.00 (s, 6H). LC/MS=212.95 (M+Na)+.

To a stirred solution of (2R)-1-[tert-butyl(dimethyl)silyl]oxypropan-2-ol (0.50 g, 2.6 mmol) and benzoic acid (0.32 g, 2.6 mmol) in DCM (10 mL) was added CDI (0.47 g, 2.9 mmol). Gas evolution was noted. The mixture was stirred at room temperature for two days. The reaction was incomplete. The mixture was heated at 40° C. under nitrogen atmosphere for 24 hours. The mixture was cooled to room temperature. The volatiles were evaporated onto silica gel (5 g) and purified via chromatography using silica gel column (12 g) and 0% to 15% EtOAc:hexane solvent gradient. The desired fractions were combined and evaporated. The recovered clear oil was consistent for [(1R)-2-[tert-butyl(dimethyl)silyl]oxy-1-methyl-ethyl] benzoate (0.241 g, 0.818 mmol, 31% Yield). 1H NMR (400 MHz, DCCl₃) δ 8.10-8.01 (m, 2H), 7.60-7.51 (m, 1H), 7.48-7.39 (m, 2H), 5.26-5.14 (m, 1H), 3.81-3.75 (m, 1H), 3.74-3.68 (m, 1H), 1.35 (d, J=6.3 Hz, 2H), 0.89-0.87 (m, 9H), 0.06 (s, 3H), 0.04 (s, 3H). LC/MS=294.97 (MH)+; 316.98 (M+Na)+.

[(1R)-2-[tert-butyl(dimethyl)silyl]oxy-1-methyl-ethyl] benzoate (0.241 g, 0.818 mmol) was dissolved in THF (5 mL) then tetrabutylammonium fluoride (1.0M in THF) (0.92 mL, 0.92 mmol) was added. The mixture was stirred for 1 hour. The volatiles were evaporated. The residue was loaded onto silica gel (5 g) and purified via chromatography using silica gel (12 g) and 0% to 100% EtOAc:hexane solvent gradient. The desired fractions were combined and evaporated. The recovered clear oil appeared was crude by 1H NMR; however, it was consistent for desired [(1R)-2-hydroxy-1-methyl-ethyl] benzoate (0.10 g, 0.55 mmol, 21% Yield) and used without further purification. 1H NMR (400 MHz, DCCl₃) δ 8.11-7.99 (m, 5H), 7.62-7.53 (m, 2H), 7.50-7.41 (m, 5H), 5.31-5.17 (m, 1H), 3.86-3.72 (m, 2H), 1.38 (d, J=6.3 Hz, 3H). LC/MS=202.89 (M+Na)+.

A 1 dram vial with screw cap and stir bar was charged with 4-(2-amino-4-chlorophenyl)-2-(tert-butoxycarbonylamino)-4-oxo-butanoic acid (0.1061 g, 0.3095 mmol), CDI (0.05521 g, 0.3405 mmol) and 1,2-dichloroethane (3 mL). The suspension was stirred for 10 minutes until a solution resulted then [(1R)-2-hydroxy-1-methyl-ethyl] benzoate (0.06135 g, 0.3405 mmol) was added and the mixture was stirred at room temperature overnight. The volatiles were evaporated onto silica gel (5 g). The mixture was purified via chromatography using silica gel column (12 g) and 0% to 80% EtOAc:hexane solvent gradient. The desired fraction were combined and evaporated to yield [(1R)-2-[4-(2-amino-4-chlorophenyl)-2-(tert-butoxycarbonylamino)-4-oxo-butanoyl]oxy-1-methyl-ethyl] benzoate. The crude material was used without further purification in the next step.

[(1R)-2-[4-(2-amino-4-chlorophenyl)-2-(tert-butoxycarbonylamino)-4-oxo-butanoyl]oxy-1-methyl-ethyl] benzoate was dissolved in DCM (0.5 mL) and TFA (0.7 g, 0.5 mL, 6 mmol) was added. The mixture was stirred at room temperature for 15 minutes. The volatiles were evaporated and the residue was purified via reverse phase chromatography using 15% to 60% ACN:water (w/0.1% TFA as modifier) solvent gradient. The desired fractions were combined, frozen and lyophilized. The recovered lyophilate was hygroscopic and was dissolved in methanol (1 mL) and evaporated (twice) then placed under high vacuum for 24 hours. The recovered yellow resin solid was consistent for desired [(1R)-2-[2-amino-4-(2-amino-4-chloro-phenyl)-4-oxo-butanoyl]oxy-1-methyl-ethyl] benzoate as the TFA salt (0.01796 g, 0.02838 mmol, 9.2% Yield). 1H NMR (400 MHz, DMSO-d6) δ 8.32 (br. s., 3H), 7.88-7.79 (m, 2H), 7.67-7.55 (m, 2H), 7.42 (t, J=7.5 Hz, 2H), 7.34 (br. s., 2H), 6.79 (dd, J=10.7, 2.1 Hz, 1H), 6.51 (ddd, J=8.7, 7.0, 2.1 Hz, 1H), 5.36-5.14 (m, 1H), 4.53-4.24 (m, 3H), 3.71-3.49 (m, 2H), 1.32-1.26 (m, 3H). LC/MS=404.95, 406.92 (MH)+; chlorine motif.

### Example 19: 3-Amino-8-chloro-3,4-dihydro-1H-1-benzazepine-2,5-dione

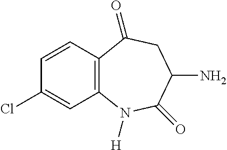 [SMILES: NC1CC(=O)c2ccc(Cl)cc2NC1=O]

A 1 dram vial with screw cap and stir bar was charged with 4-(2-amino-4-chlorophenyl)-2-(tert-butoxycarbonylamino)-4-oxo-butanoic acid (0.0750 g, 0.219 mmol) and DMF (1 mL) followed by the addition of DCC (1.0M in DCM) (0.24 mL, 0.241 mmol). The mixture was stirred at room temperature overnight. The reaction mixture was diluted with water (5 mL) and extracted with EtOAc (25 mL). The organic was washed with water (2×10 mL) and saturated aqueous NaCl (5 mL). The organic was dried over magnesium sulfate, filtered and evaporated. The residue was purified via chromatography using silica gel (12 g) and 0% to 100% EtOAc:hexane. The desired fractions were combined and evaporated to a pale yellow resin that was crude by 1H NMR. The crude resin was dissolved in DCM (0.5 mL) then TFA (0.5 mL) was added. The mixture was stirred for 15 minutes. The volatiles were evaporated and the residue was purified via reverse phase chromatography using 0% to 40% ACN:water (w/0.1% TFA as modifier) solvent gradient. The desired fractions were combined, frozen and lyophilized. The recovered white lyophilate was consistent for 3-amino-8-chloro-3,4-dihydro-1H-1-benzazepine-2,5-dione; TFA (0.0085 g, 0.025 mmol, 11% Yield). 1H NMR (400 MHz, DMSO-d6) δ 10.90 (br. s, 1H), 8.40 (br. s., 3H), 7.86 (d, J=8.5 Hz, 1H), 7.36 (dd, J=8.5, 2.0 Hz, 1H), 7.28 (d, J=2.0 Hz, 1H), 4.68 (dd, J=13.6, 2.3 Hz, 1H), 3.40-3.27 (m, 1H), 2.98 (dd, J=17.8, 2.3 Hz, 1H). LC/MS=225.01, 227.00 (MH)+; chlorine motif.

### Example 20: Benzyl 2-amino-4-(2-amino-4-chlorophenyl)-4-oxo-butanoate

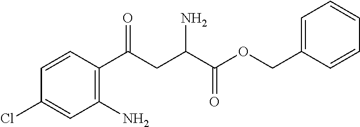 [SMILES: Nc1cc(Cl)ccc1C(=O)CC(N)C(=O)OCc1ccccc1]

A 1 dram vial with screw cap and stir bar was charged with 4-(2-amino-4-chlorophenyl)-2-(tert-butoxycarbonylamino)-4-oxo-butanoic acid (0.0772 g, 0.225 mmol), 1,2-dichloroethane (1 mL) then CDI (0.0414 g, 0.255 mmol). The suspension was stirred at room temperature until a solution resulted then benzyl alcohol (0.0278 g, 0.0266 mL, 0.257 mmol) was added. The mixture was stirred at room temperature overnight. The reaction was complete. To the stirred solution was added TFA (1.0 mL) and the mixture was stirred for 15 minutes. The volatiles were evaporated. The residue was purified via reverse phase chromatography using 15% to 60% ACN:water (w/0.1% TFA as modifier) solvent gradient. The desired fractions were combined, frozen and lyophilized. The recovered pale yellow lyophilate was consistent for desired benzyl 2-amino-4-(2-amino-4-chlorophenyl)-4-oxo-butanoate as the TFA salt (0.0300 g, 0.0535 mmol, 23.8% Yield). 1H NMR (400 MHz, DMSO-d6) δ 8.39 (br. s., 3H), 7.74 (d, J=8.8 Hz, 1H), 7.58-7.24 (m, 7H), 6.88 (d, J=2.0 Hz, 1H), 6.59 (dd, J=8.7, 2.1 Hz, 1H), 5.22 (s, 2H), 4.52 (t, J=4.6 Hz, 1H), 3.75-3.55 (m, 2H). LC/MS=333.08, 335.07 (MH)+; chlorine motif.

### Example 21: Butyl 2-acetamido-4-(2-amino-4-chlorophenyl)-4-oxo-butanoate

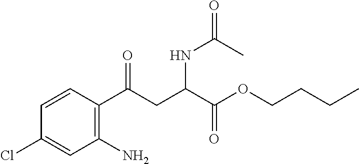 [SMILES: CCCCOC(=O)C(CC(=O)c1ccc(Cl)cc1N)NC(C)=O]

A 1 dram vial with screw cap and stir bar was charged with 2-acetamido-4-(2-amino-4-chlorophenyl)-4-oxo-butanoic acid (0.1000 g, 0.3512 mmol) and 1,2-dichloroethane (1 mL). To the stirred suspension was added CDI (0.06835 g, 0.4215 mmol). The suspension was stirred for 30 minutes. 1-Butanol (0.02864 g, 0.0354 mL, 0.3864 mmol) was added to the suspension and the mixture was stirred at room temperature overnight. The red suspension was acidified with TFA (0.5 mL) and the volatiles were evaporated. The residue was purified via reverse phase chromatography using 20% to 65% ACN:water (w/0.1% TFA as modifier) solvent gradient. The desired fractions were combined, frozen and lyophilized. The recovered pale yellow lyophilate was consistent for desired butyl 2-acetamido-4-(2-amino-4-chloro-phenyl)-4-oxo-butanoate as the TFA salt (0.0281 g, 0.0618 mmol, 17.6% Yield). 1H NMR (400 MHz, DMSO-d6) δ 8.22 (d, J=7.8 Hz, 1H), 7.75 (d, J=8.8 Hz, 1H), 6.84 (d, J=2.3 Hz, 1H), 6.56 (dd, J=8.8, 2.0 Hz, 1H), 4.77-4.67 (m, 1H), 4.08-3.96 (m, 2H), 3.36 (dd, J=6.0, 1.8 Hz, 2H), 1.82 (s, 3H), 1.55-1.45 (m, 2H), 1.33-1.22 (m, 2H), 0.84 (t, J=7.3 Hz, 3H). LC/MS=341.09, 343.08 (MH)+; chlorine motif.

### Example 22: 2-Amino-4-(2-amino-4-chlorophenyl)-1-[(2R)-2-methylpyrrolidin-1-yl]butane-1,4-dione

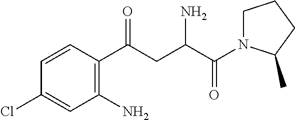 [SMILES: C[C@@H]1CCCN1C(=O)C(N)CC(=O)c1ccc(Cl)cc1N]

A 1 dram vial with screw cap and stir bar was charged with 4-(2-amino-4-chlorophenyl)-2-(tert-butoxycarbonylamino)-4-oxo-butanoic acid (0.1000 g, 0.2917 mmol) and 1,2-dichloroethane (1 mL) followed by CDI (0.05676 g, 0.3501 mmol) and stirred until a yellow solution resulted. (2R)-2-methylpyrrolidine (0.02732 g, 0.3209 mmol) was added and the mixture was stirred at room temperature overnight. The reaction was complete by LC/MS and consistent for tert-butyl N-[3-(2-amino-4-chlorophenyl)-1-[(2R)-2-methylpyrrolidine-1-carbonyl]-3-oxo-propyl]carbamate. To the yellow solution was added TFA (0.5 mL). The mixture was stirred for 15 minutes. The volatiles were evaporated and the residue was purified via reverse phase chromatography using 10% to 50% ACN:water (w/0.1% TFA as modifier) solvent gradient. The desired fractions were combined, frozen and lyophilized. The recovered pale yellow lyophilate was consistent for desired 2-amino-4-(2-amino-4-chlorophenyl)-1-[(2R)-2-methylpyrrolidin-1-yl]butane-1,4-dione as the TFA salt (0.0227 g, 0.0422 mmol, 14.5% Yield). 1H NMR (400 MHz, DMSO-d6) δ 8.38-7.97 (m, 3H), 7.79-7.69 (m, 1H), 7.66-7.14 (m, 2H), 6.87 (d, J=2.0 Hz, 1H), 6.60 (s, 1H), 4.46 (br. s., 1H), 4.14-3.95 (m, 1H), 3.62-3.28 (m, 4H), 2.03-1.77 (m, 3H), 1.74-1.47 (m, 1H), 1.21-1.06 (m, 3H). LC/MS=310.10, 312.10 (MH)+; chlorine motif.

### Example 23: 2-Amino-4-(2-amino-4-chlorophenyl)-1-[(2S)-2-methylpyrrolidin-1-yl]butane-1,4-dione

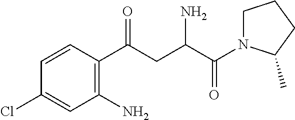 [SMILES: C[C@H]1CCCN1C(=O)C(N)CC(=O)c1ccc(Cl)cc1N]

A 1 dram vial with screw cap and stir bar was charged with 4-(2-amino-4-chloro-phenyl)-2-(tert-butoxycarbonylamino)-4-oxo-butanoic acid (0.1000 g, 0.2917 mmol) and 1,2-dichloroethane (1 mL) followed by CDI (0.05676 g, 0.3501 mmol) and stirred until a yellow solution resulted. (2S)-2-methylpyrrolidine (0.02732 g, 0.3209 mmol) was added and the mixture was stirred at room temperature overnight. The reaction was complete by LC/MS and consistent for tert-butyl N-[3-(2-amino-4-chlorophenyl)-1-[(2S)-2-methylpyrrolidine-1-carbonyl]-3-oxo-propyl]carbamate. To the yellow solution was added TFA (0.5 mL). The mixture was stirred for 15 minutes. The volatiles were evaporated and the residue was purified via reverse phase chromatography using 10% to 50% ACN:water (w/0.1% TFA as modifier) solvent gradient. The desired fractions were combined, frozen and lyophilized. The recovered pale yellow lyophilate was consistent for desired 2-amino-4-(2-amino-4-chlorophenyl)-1-[(2S)-2-methyl-pyrrolidin-1-yl]butane-1,4-dione as the TFA salt (0.0227 g, 0.0422 mmol, 14.5% Yield). 1H NMR (400 MHz, DMSO-d6) δ 8.31-8.01 (m, 3H), 7.79-7.68 (m, 1H), 7.44 (br. s., 2H), 6.87 (d, J=1.8 Hz, 1H), 6.59 (dt, J=8.6, 2.6 Hz, 1H), 4.56-4.36 (m, 1H), 4.16-3.92 (m, 1H), 3.81-3.16 (m, 4H), 2.06-1.77 (m, 3H), 1.73-1.46 (m, 1H), 1.23-1.03 (m, 3H). LC/MS=310.10, 312.09 (MH)+; chlorine motif.

### Example 24: 2-Amino-4-(2-amino-4-chlorophenyl)-N-(2-dimethylaminoethyl)-4-oxo-butanamide

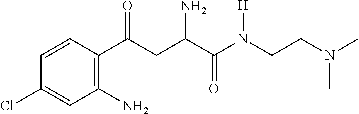 [SMILES: CN(C)CCNC(=O)C(N)CC(=O)c1ccc(Cl)cc1N]

A 1 dram vial with screw cap and stir bar was charged with 4-(2-amino-4-chlorophenyl)-2-(tert-butoxycarbonylamino)-4-oxo-butanoic acid (0.1022 g, 0.2981 mmol) and 1,2-dichloroethane (1 mL,) followed by CDI (0.05801 g, 0.3578 mmol). The mixture was stirred until a yellow solution resulted. N′,N′-dimethylethane-1,2-diamine (0.02891 g, 0.3279 mmol) was added and the mixture was stirred at room temperature overnight. The reaction was complete by LC/MS and product was consistent for tert-butyl N-[3-(2-amino-4-chlorophenyl)-1-(2-dimethylaminoethylcarbamoyl)-3-oxo-propyl]carbamate. To the solution was added TFA (0.7 g, 0.5 mL, 6 mmol). The mixture was stirred at room temperature for 15 minutes. The volatiles were evaporated. The residue was purified via reverse phase chromatography using 0% to 40% ACN:water (w/0.1% TFA as modifier) solvent gradient. The desired fractions were combined, frozen and lyophilized. The recovered pale yellow lyophilate was consistent for 2-amino-4-(2-amino-4-chlorophenyl)-N-(2-dimethylaminoethyl)-4-oxo-butanamide as the TFA salt (0.1033 g, 0.1910 mmol, 64.07% Yield). 1H NMR (400 MHz, DMSO-d6) δ 9.76 (br. s, 1H), 8.68 (t, J=5.6 Hz, 1H), 8.19 (br. s., 3H), 7.73 (d, J=8.8 Hz, 1H), 7.65-7.28 (m, 2H), 6.89 (d, J=2.3 Hz, 1H), 6.60 (dd, J=8.8, 2.0 Hz, 1H), 4.30-4.16 (m, 1H), 3.56-3.34 (m, 4H), 3.22-3.09 (m, 2H), 2.82 (s, 6H). LC/MS=313.14, 315.11 (MH)+; chlorine motif.

### Example 25: Hexyl 2-amino-4-(2-amino-4-chlorophenyl)-4-oxo-butanoate

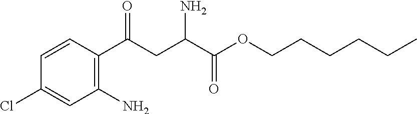 [SMILES: CCCCCCOC(=O)C(N)CC(=O)c1ccc(Cl)cc1N]

A 1 dram vial with screw cap and stir bar was charged with 4-(2-amino-4-chlorophenyl)-2-(tert-butoxycarbonylamino)-4-oxo-butanoic acid (0.1000 g, 0.2917 mmol) and 1,2-dichloroethane (1 mL). The suspension was stirred at room temperature and CDI (0.05676 g, 0.3501 mmol) was added and was stirred for 15 minutes until a yellow solution resulted. 1-Hexanol (0.03279 g, 0.0402 mL, 0.3209 mmol) was added and the mixture was stirred at room temperature overnight. The reaction was complete by LC/MS. TFA (0.5 mL) was added and the mixture was stirred at room temperature for 20 minutes. The volatiles were evaporated and the residue was subjected to high vacuum for 30 minutes. The residue was dissolved in ACN (1 mL) and methanesulfonic acid (2 equivalents) was added. A suspension resulted within 1-2 minutes. The mixture was stirred for 15 minutes then filtered, rinsed with acetonitrile and partially dried by suction. The solid was subjected to high vacuum for 3 hours. The recovered off-white solid was consistent for hexyl 2-amino-4-(2-amino-4-chloro-phenyl)-4-oxo-butanoate as the bis-methanesulfonic acid salt (0.0510 g, 0.0983 mmol, 33.7% Yield). 1H NMR (400 MHz, DMSO-d6) δ 8.29 (d, J=4.3 Hz, 3H), 7.76 (d, J=8.8 Hz, 1H), 7.68-7.14 (m, 2H), 6.88 (d, J=2.0 Hz, 1H), 6.60 (dd, J=8.7, 2.1 Hz, 1H), 4.48-4.39 (m, 1H), 4.25-3.89 (m, 2H), 3.74-3.50 (m, 2H), 2.31 (s, 6H), 1.60-1.42 (m, 2H), 1.27-1.09 (m, 6H), 0.79 (s, 3H). LC/MS=327.12, 329.10 (MH)+; chlorine motif.

### Example 26: Octyl 2-amino-4-(2-amino-4-chlorophenyl)-4-oxo-butanoate

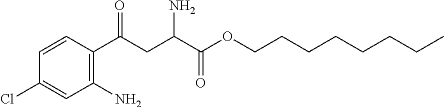 [SMILES: CCCCCCCCOC(=O)C(N)CC(=O)c1ccc(Cl)cc1N]

A 1 dram vial with screw cap and stir bar was charged with 4-(2-amino-4-chlorophenyl)-2-(tert-butoxycarbonylamino)-4-oxo-butanoic acid (0.1000 g, 0.2917 mmol) and 1,2-dichloroethane (1 mL). The suspension was stirred at room temperature and CDI (0.05676 g, 0.3501 mmol) was added and was stirred for 15 minutes until yellow solution resulted. 1-Octanol (0.04179 g, 0.0507 mL, 0.3209 mmol) was added and the mixture was stirred at room temperature overnight. The reaction was complete by LC/MS. TFA (0.5 mL) was added. The mixture was stirred at room temperature for 20 minutes. The volatiles were evaporated and the residue was subjected to high vacuum for 30 minutes. The residue was dissolved in ACN (1 mL) and methanesulfonic acid (2 equivalents) was added. A suspension results within 1-2 minutes. The mixture was stirred for 15 minutes then filtered, rinsed with ACN and partially dried by suction. The solid was subjected to high vacuum for 3 hours. The recovered off-white solid was consistent for octyl 2-amino-4-(2-amino-4-chlorophenyl)-4-oxo-butanoate as the bis-methanesulfonic acid salt (0.0694 g, 0.127 mmol, 43.5% Yield). 1H NMR (400 MHz, DMSO-d6) δ 8.29 (d, J=4.3 Hz, 3H), 7.76 (d, J=8.8 Hz, 1H), 7.66-7.20 (m, 1H), 6.87 (d, J=2.3 Hz, 1H), 6.59 (dd, J=8.7, 2.1 Hz, 1H), 4.43 (d, J=4.5 Hz, 2H), 4.19 (dd, J=10.8, 6.3 Hz, 2H), 4.09-4.01 (m, 1H), 3.73-3.49 (m, 2H), 2.32 (s, 6H), 1.51 (d, J=4.3 Hz, 2H), 1.27-1.06 (m, 10H), 0.83 (t, J=7.2 Hz, 3H). LC/MS=355.15, 357.14 (MH)+; chlorine motif.

### Example 27: 2-Amino-4-(2-amino-4-chlorophenyl)-N,N-dimethyl-4-oxo-butanamide

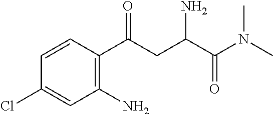 [SMILES: CN(C)C(=O)C(N)CC(=O)c1ccc(Cl)cc1N]

A 1 dram vial with screw cap and stir bar was charged with 4-(2-amino-4-chlorophenyl)-2-(tert-butoxycarbonylamino)-4-oxo-butanoic acid (0.1019 g, 0.2973 mmol) and 1,2-dichloroethane (1 mL). The suspension was stirred vigorously and CDI (0.0651 g, 0.401 mmol) was added and was stirred for 10 minutes until yellow solution resulted. Dimethylamine (2.0M in THF) (0.22 mL, 0.44 mmol) was added. The mixture was stirred at room temperature overnight. The reaction was complete by LC/MS. TFA (0.5 mL) was added and the mixture was stirred at room temperature for 30 minutes. The volatiles were evaporated. The residue was purified via reverse phase chromatography using 10% to 50% acetonitrile:water (w/0.1% TFA as modifier) solvent gradient. The desired fractions were combined, frozen and lyophilized. The recovered pale yellow lyophilate was consistent for desired 2-amino-4-(2-amino-4-chloro-phenyl)-N,N-dimethyl-4-oxo-butanamide as the TFA salt (0.0945 g, 0.190 mmol, 63.9% Yield). 1H NMR (400 MHz, DMSO-d6) δ 8.07 (br. s., 3H), 7.74 (d, J=8.8 Hz, 1H), 7.58-7.28 (m, 2H), 6.88 (d, J=2.0 Hz, 1H), 6.59 (dd, J=8.8, 2.3 Hz, 1H), 4.71 (br. s., 1H), 3.53-3.31 (m, 2H), 2.99 (s, 3H), 2.90 (s, 3H). LC/MS=270.07, 272.07 (MH)+; chlorine motif.

### Example 28: 2-Amino-4-(2-amino-4-chlorophenyl)-N-methylsulfonyl-4-oxo-butanamide

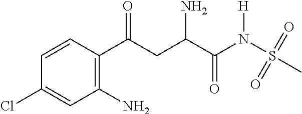 [SMILES: CS(=O)(=O)NC(=O)C(N)CC(=O)c1ccc(Cl)cc1N]

A 1 dram vial with screw cap and stir bar was charged with 4-(2-amino-4-chlorophenyl)-2-(tert-butoxycarbonylamino)-4-oxo-butanoic acid (0.100 g, 0.292 mmol) and 1,2-dichloroethane (1 mL). To the vigorously stirred suspension was added CDI (0.0597 g, 0.368 mmol) and the mixture was stirred for 10 minutes until a yellow solution resulted. Methanesulfonamide (0.0348 g, 0.366 mmol) was added followed by 1,8-diazabicyclo[5.4.0]-undec-7-ene (0.0545 g, 0.0540 mL, 0.351 mmol). The slowly darkening mixture was stirred at room temperature overnight. The reaction was complete. To the dark solution was added TFA (0.5 mL) and the mixture was stirred at room temperature for 30 minutes. The volatiles were evaporated. The residue was purified via reverse phase chromatography using 10% to 50% ACN:water (w/0.1% TFA as modifier) solvent gradient. The desired fractions were combined, frozen and lyophilized. The recovered pale yellow lyophilate was consistent for desired 2-amino-4-(2-amino-4-chlorophenyl)-N-methylsulfonyl-4-oxo-butanamide as the TFA salt (0.0538 g, 0.0982 mmol, 33.7% Yield). 1H NMR (400 MHz, DMSO-d6) δ 8.18 (br. s., 3H), 7.77 (d, J=8.8 Hz, 1H), 7.69-7.12 (m, 2H), 6.89-6.88 (m, 1H), 6.60 (dd, J=8.8, 2.3 Hz, 1H), 4.35-4.19 (m, 1H), 3.68-3.24 (m, 2H), 3.20 (s, 3H). LC/MS=320.02, 322.01 (MH)+; chlorine motif.

### Example 29: 2-Amino-4-(2-amino-4-chlorophenyl)-N-isopropylsulfonyl-4-oxo-butanamide

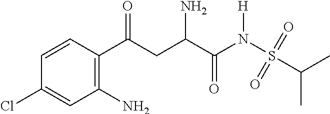 [SMILES: CC(C)S(=O)(=O)NC(=O)C(N)CC(=O)c1ccc(Cl)cc1N]

A 1 dram vial with screw cap and stir bar was charged with 4-(2-amino-4-chlorophenyl)-2-(tert-butoxycarbonylamino)-4-oxo-butanoic acid (0.1010 g, 0.2946 mmol) and 1,2-dichloroethane (1 mL). To the vigorously stirred suspension was added CDI (0.0637 g, 0.393 mmol) and the was stirred 10 minutes until a yellow solution was resulted. Propane-2-sulfonamide (0.0399 g, 0.324 mmol) was added followed by 1,8-diazabicyclo[5.4.0]undec-7-ene (0.0545 g, 0.0540 mL, 0.351 mmol). The slowly darkening mixture was stirred at room temperature overnight. The reaction was complete. TFA (0.5 mL) was added and the mixture was stirred at room temperature for 30 minutes. The volatiles were evaporated. The residue was purified via reverse phase chromatography using 10% to 50% ACN:water (w/0.1% TFA as modifier) solvent gradient. The desired fractions were combined, frozen and lyophilized. The recovered pale yellow lyophilate was consistent for desired 2-amino-4-(2-amino-4-chloro-phenyl)-N-isopropylsulfonyl-4-oxo-butanamide as the TFA salt (0.0254 g, 0.0441 mmol, 15.0% Yield). 1H NMR (400 MHz, DMSO-d6) δ 8.19 (br. s., 3H), 7.78 (d, J=8.8 Hz, 1H), 7.72-7.06 (m, 2H), 6.88 (d, J=2.0 Hz, 1H), 6.60 (dd, J=8.8, 2.3 Hz, 1H), 4.30 (br. s., 1H), 3.70-3.52 (m, 3H), 1.31-1.23 (m, 6H). LC/MS=348.03, 350.04 (MH)+; chlorine motif.

### Example 30: 2-[[2-amino-4-(2-amino-4-chlorophenyl)-4-oxo-butanoyl]-amino]acetic acid

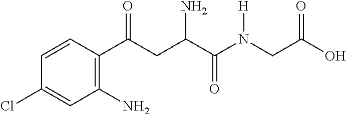 [SMILES: Nc1cc(Cl)ccc1C(=O)CC(N)C(=O)NCC(=O)O]

A 1 dram vial with screw cap and stir bar was charged with 4-(2-amino-4-chloro-phenyl)-2-(tert-butoxycarbonylamino)-4-oxo-butanoic acid (0.1011 g, 0.2949 mmol) and 1,2-dichloroethane (1.256 g, 1 mL, 12.57 mmol). To the vigorously stirred suspension was added CDI (0.0564 g, 0.348 mmol) and was stirred for 10 minutes until a yellow solution resulted. TEA (0.0603 g, 0.0830 mL, 0.590 mmol) was added followed by tert-butyl 2-aminoacetate HCl (0.0149 g, 0.0889 mmol). The mixture was stirred at room temperature overnight. The reaction was complete by LC/MS. To the suspension was added anisole (0.0645 g, 0.0650 mL, 0.597 mmol) followed by TFA (1 mL). The mixture was stirred for 1 hour until complete deprotection was observed by LC/MS and HPLC. The volatiles were evaporated. The dark residue was purified via reverse phase chromatography using 0% to 40% ACN:water (w/0.1% TFA as modifier) solvent gradient. The desired fractions were combined, frozen and lyophilized. The recovered pale yellow lyophilate was consistent for desired 2-[[2-amino-4-(2-amino-4-chloro-phenyl)-4-oxo-butanoyl]amino]acetic acid as the TFA salt (0.0785 g, 0.149 mmol, 50.4% Yield). 1H NMR (400 MHz, DMSO-d6) δ 13.57-11.95 (m, 1H), 8.72 (t, J=5.6 Hz, 1H), 8.59-7.80 (m, 3H), 7.71 (d, J=8.8 Hz, 1H), 7.55-7.32 (m, 2H), 6.88 (d, J=2.0 Hz, 1H), 6.60 (dd, J=8.7, 2.1 Hz, 1H), 4.31 (dd, J=7.0, 4.5 Hz, 1H), 3.97-3.77 (m, 2H), 3.58-3.43 (m, 2H). LC/MS=300.03, 302.05 (MH)+; chlorine motif.

### Example 31: [(2R,3R,4S,5R,6S)-3,4,5,6-tetraacetoxytetrahydropyran-2-yl]methyl 4-(2-amino-4-chlorophenyl)-2-(tert-butoxycarbonylamino)-4-oxo-butanoate

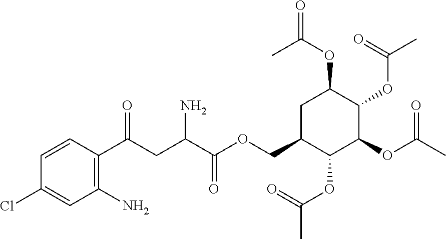 [SMILES: CC(=O)O[C@H]1[C@H](OC(C)=O)[C@@H](COC(=O)C(N)CC(=O)c2ccc(Cl)cc2N)C[C@@H](OC(C)=O)[C@@H]1OC(C)=O]

To a suspension of 4-(2-amino-4-chloro-phenyl)-2-(tert-butoxycarbonylamino)-4-oxo-butanoic acid (0.1000 g, 0.2917 mmol) in 1,2-dichloroethane (1 mL) was added CDI (0.05364 g, 0.3209 mmol) and was stirred at room temperature for 10 minutes until a clear yellow solution resulted. [(2R,3R,4S,5R,6S)-4,5,6-triacetoxy-2-(hydroxymethyl)tetrahydropyran-3-yl] acetate (0.1219 g, 0.3501 mmol) was added and the mixture was stirred at room temperature overnight. The reaction mixture was evaporated onto a silica gel (5 g) and purified via chromatography using silica gel column (12 g) and 0% to 60% EtOAc:hexane solvent gradient. The desired fractions were combined and evaporated to a yellow sticky resin. The material was subjected to high vacuum for 2 hours. The recovered yellow foam was consistent for [(2R,3R,4S,5R,6S)-3,4,5,6-tetraacetoxytetrahydropyran-2-yl]methyl (2S)-4-(2-amino-4-chlorophenyl)-2-(tert-butoxycarbonylamino)-4-oxo-butanoate (0.115 g, 0.171 mmol, 58.6% Yield). 1H NMR (400 MHz, DMSO-d6) δ 7.72 (dd, J=8.8, 4.8 Hz, 1H), 7.38 (br. s., 2H), 7.11 (dd, J=11.9, 7.9 Hz, 1H), 6.84 (d, J=1.8 Hz, 1H), 6.56 (dd, J=8.8, 2.0 Hz, 1H), 5.93 (dd, J=8.4, 3.1 Hz, 1H), 5.46-5.37 (m, 1H), 5.05-4.90 (m, 2H), 4.60-4.45 (m, 1H), 4.26-4.06 (m, 3H), 3.49-3.12 (m, 2H), 2.06-1.91 (m, 12H), 1.37 (d, J=3.0 Hz, 9H). LC/MS=695.18, 697.18 (M+Na)+; chlorine motif.

To a solution of [(2R,3R,4S,5R,6S)-3,4,5,6-tetraacetoxytetrahydropyran-2-yl]methyl (2S)-4-(2-amino-4-chlorophenyl)-2-(tert-butoxycarbonylamino)-4-oxo-butanoate (0.115 g, 0.171 mmol) in DCM (1 mL) was added TFA (1 mL, 13.0 mmol) was added. The mixture was stirred for 15 minutes until reaction was complete. The volatiles were evaporated. The residue was purified via reverse phase chromatography using 10% to 55% ACN:water (w/0.1% TFA as modifier) solvent gradient. The desired fractions were combined, frozen and lyophilized. The recovered pale yellow lyophilate was consistent for desired [(2R,3R,4S,5R,6S)-3,4,5,6-tetra-acetoxytetrahydropyran-2-yl]methyl 2-amino-4-(2-amino-4-chlorophenyl)-4-oxo-butanoate as the TFA salt (0.0900 g, 0.131 mmol, 44.9% Yield). 1H NMR (400 MHz, DMSO-d6) δ 8.54-8.10 (m, 3H), 7.79-7.69 (m, 1H), 7.65-7.14 (m, 2H), 6.88 (t, J=2.0 Hz, 1H), 6.61 (dd, J=8.5, 1.8 Hz, 1H), 5.92 (dd, J=8.3, 4.0 Hz, 1H), 5.43 (td, J=9.5, 2.3 Hz, 1H), 5.10 (td, J=9.7, 6.8 Hz, 1H), 4.96 (dt, J=9.6, 8.0 Hz, 1H), 4.55-4.35 (m, 1H), 4.33-4.15 (m, 3H), 3.67-3.53 (m, 2H), 2.05-1.89 (m, 12H). LC/MS=573.11, 575.09 (MH)+; chlorine motif.

### Example 32: tert-Butyl (2S)-2-[[2-amino-4-(2-amino-4-chlorophenyl)-4-oxo-butanoyl]amino]-3-methyl-butanoate

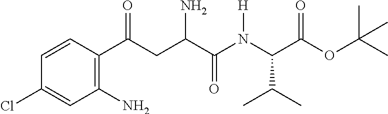 [SMILES: CC(C)[C@H](NC(=O)C(N)CC(=O)c1ccc(Cl)cc1N)C(=O)OC(C)(C)C]

A 1 dram vial screw cap and stir bar was charged with 4-(2-amino-4-chloro-phenyl)-2-(tert-butoxycarbonylamino)-4-oxo-butanoic acid (0.1067 g, 0.3113 mmol) and suspended in 1,2-dichloroethane (1 mL). CDI (0.0755 g, 0.466 mmol) was added and was stirred for 15 minutes until a yellow solution resulted. tert-Butyl (2S)-2-amino-3-methyl-butanoate HCl (0.0812 g, 0.387 mmol) and TEA (0.0653 g, 0.09 mL, 0.639 mmol) were added. The mixture was stirred at room temperature overnight. The reaction mixture was evaporated onto silica gel column (12 g) and purified via chromatography using 0% to 70% EtOAc:hexane solvent gradient. The desired fractions were combined and evaporated. The recovered yellow resin was consistent for desired intermediate tert-butyl (2S)-2-[[4-(2-amino-4-chlorophenyl)-2-(tert-butoxycarbonylamino)-4-oxo-butanoyl]amino]-3-methyl-butanoate (0.0837 g, 0.168 mmol, 54.0% Yield) by LC/MS [498.10, 500.09 (MH)+; chlorine motif].

tert-Butyl (2S)-2-[[4-(2-amino-4-chlorophenyl)-2-(tert-butoxycarbonylamino)-4-oxo-butanoyl]amino]-3-methyl-butanoate (0.0837 g, 0.168 mmol) was dissolved in DCM (1 mL) and THF (1 mL) then p-toluenesulfonic acid monohydrate (0.25 g, 0.202 mL, 1.29 mmol) was added. The mixture was stirred at room temperature overnight. Reaction was complete by LC/MS. The mixture was loaded onto Phenomenex SX-C cartridge (2 g) and washed with methanol (2×10 mL) to remove p-TSA then the product was released with 2M ammonia in methanol (10 mL). The filtrate was evaporated. The residue was purified via reverse phase chromatography using 15% to 60% ACN:water (w/0.1% TFA as modifier) solvent gradient. The desired fractions were combined, frozen and lyophilized. The recovered pale yellow lyophilate was consistent for tert-butyl(2S)-2-[[2-amino-4-(2-amino-4-chlorophenyl)-4-oxo-butanoyl]amino]-3-methylbutanoate as the TFA salt (0.0181 g, 0.0354 mmol, 11.4% Yield). 1H NMR (400 MHz, DMSO-d6) δ 8.55 (dd, J=16.9, 8.4 Hz, 1H), 8.10 (br. s., 3H), 7.78-7.63 (m, 1H), 7.57-7.34 (m, 2H), 6.89 (dd, J=6.1, 2.1 Hz, 1H), 6.61 (td, J=8.7, 2.3 Hz, 1H), 4.49-4.26 (m, 1H), 4.16 (ddd, J=15.4, 8.3, 5.4 Hz, 1H), 3.64-3.20 (m, 2H), 2.19-1.98 (m, 1H), 1.42 (d, J=8.8 Hz, 9H), 0.97-0.80 (m, 6H). LC/MS=398.12, 400.14 (MH)+; chlorine motif.

### Example 33: tert-Butyl (2S)-2-[[2-amino-4-(2-amino-4-chlorophenyl)-4-oxo-butanoyl]amino]-3-(4-hydroxyphenyl)propanoate

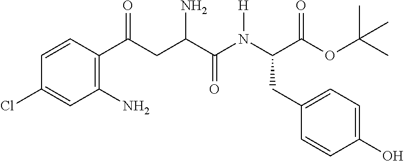 [SMILES: CC(C)(C)OC(=O)[C@H](Cc1ccc(O)cc1)NC(=O)C(N)CC(=O)c1ccc(Cl)cc1N]

A 1 dram vial screw cap and stir bar was charged with 4-(2-amino-4-chloro-phenyl)-2-(tert-butoxycarbonylamino)-4-oxo-butanoic acid (0.1000 g, 0.2917 mmol) and suspended in 1,2-dichloroethane (1 mL). CDI (0.05203 g, 0.3209 mmol) was added and was stirred for 15 minutes until a yellow solution resulted. tert-Butyl (2S)-2-amino-3-(4-hydroxyphenyl)propanoate HCl (0.09585 g, 0.3501 mmol) and TEA (0.0653 g, 0.09 mL, 0.639 mmol) were added. The mixture was stirred at room temperature overnight. The reaction mixture was loaded onto silica gel and purified via chromatography using 0% to 70% EtOAc:hexane solvent gradient. The desired fractions were combined and evaporated. The recovered yellow resin was consistent for desired intermediate tert-butyl (2S)-2-[[4-(2-amino-4-chlorophenyl)-2-(tert-butoxycarbonylamino)-4-oxo-butanoyl]amino]-3-(4-hydroxyphenyl)propanoate (0.0581 g, 0.103 mmol, 35.4% Yield) by LC/MS [562.15, 564.13 (MH)+; chlorine motif].

tert-Butyl (2S)-2-[[4-(2-amino-4-chlorophenyl)-2-(tert-butoxycarbonylamino)-4-oxo-butanoyl]amino]-3-(4-hydroxyphenyl)propanoate (0.0581 g, 0.103 mmol) was dissolved in DCM (1 mL) and THF (1 mL) then p-Toluenesulfonic acid monohydrate (0.25 g, 0.202 mL, 1.29 mmol) was added. The mixture was stirred at room temperature overnight. Reaction was complete by LC/MS. The mixture was loaded onto Phenomenex SX-C cartridge (2 g) and washed with methanol (2×10 mL) to remove p-TSA then the product was released with 2M ammonia in methanol (10 mL). The filtrate was evaporated. The residue was purified via reverse phase chromatography using 15% to 60% ACN:water (w/0.1% TFA as modifier) solvent gradient. The desired fractions were combined, frozen and lyophilized. The recovered pale yellow lyophilate was consistent for tert-butyl (2S)-2-[[2-amino-4-(2-amino-4-chlorophenyl)-4-oxo-butanoyl]amino]-3-(4-hydroxyphenyl)propanoate as the TFA salt (0.0070 g, 0.012 mmol, 4.2% Yield). 1H NMR (400 MHz, DMSO-d6) δ 9.38-9.22 (m, 1H), 8.69 (dd, J=13.2, 7.9 Hz, 1H), 8.16-7.93 (m, 3H), 7.68-7.38 (m, 3H), 7.01 (dd, J=10.5, 8.5 Hz, 2H), 6.89 (d, J=2.0 Hz, 1H), 6.72-6.60 (m, 3H), 4.61-4.29 (m, 1H), 4.22 (br. s., 1H), 3.52-3.30 (m, 1H), 3.18-3.05 (m, 1H), 3.04-2.87 (m, 1H), 2.85-2.69 (m, 1H), 1.37 (d, J=19.8 Hz, 9H). LC/MS=462.15, 464.12 (MH)+; chlorine motif.

### Example 34: Methyl 4-(2-acetamido-4-chlorophenyl)-2-amino-4-oxo-butanoate

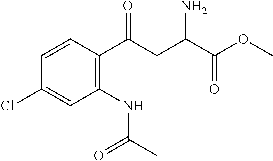 [SMILES: COC(=O)C(N)CC(=O)c1ccc(Cl)cc1NC(C)=O]

A 1 dram vial with stir bar was charged with methyl 4-(2-amino-4-chloro-phenyl)-2-(tert-butoxycarbonylamino)-4-oxo-butanoate (0.0950 g, 0.266 mmol), 4-dimethylaminopyridine (DMAP)(0.00163 g, 0.0133 mmol) and DCM (1 mL). The solution was stirred then TEA (0.0327 g, 0.0450 mL, 0.320 mmol) was added followed by acetic anhydride (0.0326 g, 0.0302 mL, 0.320 mmol). The mixture stirred at room temperature overnight. No reaction was observed. Additional acetic anhydride (0.0326 g, 0.0302 mL, 0.320 mmol) was added. The mixture was stirred for 24 hours. No reaction was observed. Acetyl chloride (2 μL) was added. The reaction was complete within 3 hours. The reaction was evaporated onto silica gel (5 g) and purified via chromatography using silica gel column (12 g) and 0% to 50% EtOAc:hexane solvent gradient. The desired fractions were combined and evaporated. The recovered resin was consistent for methyl 4-(2-acetamido-4-chloro-phenyl)-2-(tert-butoxycarbonylamino)-4-oxo-butanoate (0.0532 g, 0.133 mmol, 50.1% Yield) by mass [299.07, 301.08 [M-(BOC)+H]+; chlorine motif]. The material was used without further purification in the next step.

Methyl 4-(2-acetamido-4-chlorophenyl)-2-(tert-butoxycarbonylamino)-4-oxo-butanoate (0.0532 g, 0.133 mmol) was dissolved in DCM (1 mL) then TFA (1.48 g, 1 mL, 13.0 mmol) was added. The mixture was stirred at room temperature for 15 minutes. The volatiles were evaporated. The residue was purified via reverse phase chromatography using 0% to 45% ACN:water (w/0.1% TFA as modifier) solvent gradient. The desired fractions were combined, frozen and lyophilized. The recovered pale yellow lyophilate was consistent for desired methyl 4-(2-acetamido-4-chlorophenyl)-2-amino-4-oxo-butanoate as the TFA salt (0.0294 g, 0.0712 mmol, 26.8% Yield). 1H NMR (400 MHz, DMSO-d6) δ 10.90 (s, 1H), 8.37 (br. s., 3H), 8.30 (d, J=2.3 Hz, 1H), 7.98 (d, J=8.5 Hz, 1H), 7.34 (dd, J=8.5, 2.3 Hz, 1H), 4.50 (t, J=5.1 Hz, 1H), 3.76 (s, 3H), 3.71 (t, J=4.6 Hz, 2H), 2.14 (s, 3H). LC/MS=299.06, 301.07 (MH)+; chlorine motif.

### Example 35: Methyl 2-amino-4-[4-chloro-2-(ethoxycarbonylamino)phenyl]-4-oxo-butanoate

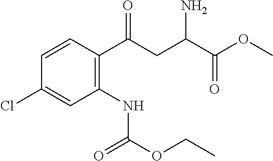 [SMILES: CCOC(=O)Nc1cc(Cl)ccc1C(=O)CC(N)C(=O)OC]

A 1 dram vial with stir bar was charged with methyl 4-(2-amino-4-chloro-phenyl)-2-(tert-butoxycarbonylamino)-4-oxo-butanoate (0.0950 g, 0.266 mmol), DMAP (0.00163 g, 0.0133 mmol) and DCM (1 mL). The solution was stirred then TEA (0.0327 g, 0.0450 mL, 0.320 mmol) was added followed by ethyl chloroformate (0.0347 g, 0.0305 mL, 0.320 mmol) was added. The mixture was stirred at room temperature over weekend. Partial reaction was observed. Additional ethyl chloroformate (0.0347 g, 0.0305 mL, 0.320 mmol) was added and stirred for 24 hours. No additional conversion was observed. The mixture was evaporated onto silica gel (5 g) and purified via chromatography using silica gel column (12 g) and 0% to 50% EtOAc:hexane solvent gradient. The desired fractions were combined and evaporated. The recovered resin was consistent for intermediate methyl 2-(tert-butoxycarbonylamino)-4-[4-chloro-2-(ethoxycarbonylamino)phenyl]-4-oxo-butanoate (0.0796 g, 0.186 mmol, 69.7% Yield) by mass [451.08, 453.07 (M+Na)+ and 329.08, 331.10 [M-(BOC)+H]+; chlorine motif]. The material was used without further purification in the next step.

Methyl 2-(tert-butoxycarbonylamino)-4-[4-chloro-2-(ethoxycarbonylamino)phenyl]-4-oxo-butanoate (0.0796 g, 0.186 mmol) was dissolved in DCM (1 mL) then TFA (1.48 g, 1 mL, 13.0 mmol) was added. The reaction mixture was stirred for 15 minutes. The volatiles were evaporated. The residue was purified via reverse phase chromatography using 5% to 50% ACN:water (w/0.1% TFA as modifier) solvent gradient. The desired fractions were combined, frozen and lyophilized. The recovered white lyophilate was consistent for desired methyl 2-amino-4-[4-chloro-2-(ethoxycarbonylamino)phenyl]-4-oxo-butanoate as the TFA salt (0.0199 g, 0.0449 mmol, 16.9% Yield). 1H NMR (400 MHz, DMSO-d6) δ 10.90 (s, 1H), 8.37 (br. s., 3H), 8.30 (d, J=2.3 Hz, 1H), 7.98 (d, J=8.5 Hz, 1H), 7.34 (dd, J=8.5, 2.3 Hz, 1H), 4.50 (t, J=5.1 Hz, 1H), 3.76 (s, 3H), 3.71 (t, J=4.6 Hz, 2H), 2.14 (s, 3H). LC/MS=329.08, 331.08 (MH)+; chlorine motif.

### Example 36

The in-life portions of the studies were conducted were approved by the Institutional Animal Care and Use Committee of Teva WC. In general, the analysis of plasma samples was conducted within 2 weeks of the collection period.

Animals

Studies were conducted in male Sprague Dawley rats. All animals were obtained from Charles River Labs (various US locations) and acclimated for at least 3 days prior to the initiation of the study.

The rats were group-housed (2-3 per cage) in micro-isolator cages in ventilated racks on Alpha-Dri bedding. They were provided ad libitum access to food (Lab Diet 5001) and water for the duration of the study. In selected studies, rats were fasted overnight prior to oral doing. House water was filtered through a reverse osmosis system (Edstrom) and pH-adjusted (2.4 to 2.7) prior to use. The facility was maintained on as 12 hour light/dark cycle (7 AM to 7 PM).

For studies in rats, the oral (i.e. PO) formulations were administered at a dose volume of 5 or 10 mL/kg using a syringe and ball-tipped stainless steel gavage needle. The i.v. dose volume in rat studies was 1 mL/kg.

Sample Collection and Processing

For the PK portion of the study, blood samples for the determination of drug concentrations were collected at pre-determined times, post dose. In rats, the samples were serially collected from a lateral tail vein into heparinized tubes. Blood was centrifuged at 4° C. and the plasma fraction was transferred into clean dry tubes and frozen on dry ice. All samples were stored at approximately −20° C. pending analysis.

Bioanalytical Methods

Plasma and tissues was prepared for high performance liquid chromatography (HPLC)/mass spectrometric analysis according to standard protocol. Following protein precipitation with acetonitrile containing an internal standard (alprenolol), the samples were analyzed for test compound and internal standard via HPLC coupled with tandem mass spectrometry. The quantifiable range of the assay was from 10 to 10000 ng/mL.

Pharmacokinetic Analysis

The PK parameters were estimated from individual rats or the composite mean of the mouse plasma concentration-versus-time data by non-compartmental analysis (Gibaldi and Perrier 1982) using WinNonlin software (Professional Version 5.2 or 6.3) Pharsight Corporation, Palo Alto, Calif., USA). The bioanalytical data were entered into a Microsoft® Excel spreadsheet.

For the calculation of the mean data, plasma concentrations below the limit of quantitation of the assay (i.e., <10 ng/mL) were designated as “BLQ” and treated as 0. Mean concentrations were reported as BLQ if the calculated value was below the lower limit of quantitation of the assay. The terminal rate constant for elimination from plasma (λ_{z}) was estimated by linear regression of the terminal portion of the semi-logarithmic plasma concentration-versus-time curve. The apparent terminal half-life (t½) was calculated as 0.693 divided by λ_{z}. C₀ was back-extrapolated by log-linear regression of the first 2 post-dose concentrations. The area under the plasma concentration-versus-time curve from time 0 to the time of the last measurable concentration (AUC₀₋ₜ) was determined by the linear trapezoidal rule. The area from zero to infinity (AUC_{0-∞}) was calculated as the sum of AUC₀₋ₜ and the area extrapolated from the last measurable concentration to infinity (Cₗₐₛₜ/λ_{Z}). The plasma clearance (CL) after iv administration was calculated as dose divided by AUC_{0-∞}, and the apparent volume of distribution (V_{d}) was calculated as dose divided by (AUC_{0-∞}·λ_{z}).

The compounds of Examples 16, 31, and 32 were tested using the above protocol at the doses listed in the table and analyzed for the presence of (+/−)-4-chlorokynurenine in plasma. The results are shown in Table 1.

TABLE 1

| | | |
|---|---|---|
| (+/−)-Chlorokynurenine | 1 mg/kg IV | 5 mg/kg PO |

Example 16

| | | |
|---|---|---|
| Cₘₐₓ (ng/mL) | 5277 ± 2589 | 661 ± 175 |
| tₘₐₓ (h) | 0.08 ± 0 | 0.25 ± 0 |
| AUC0-t (ng * h/mL) | 2451 ± 1442 | 1359 ± 243 |
| AUC_{0-∞} (ng * h/mL) | 2533 ± 1448 | 1680 ± 206 |
| t½ (h) | 0.5 ± 0.1 | 2.3 ± 0.2 |

Example 31

| | | |
|---|---|---|
| Cₘₐₓ (ng/mL) | 384 ± 66 | 225 ± 32 |
| tₘₐₓ (h) | 0.3 ± 0.1 | 0.3 ± 0.1 |
| AUC0-t (ng * h/mL) | 296 ± 36 | 321 ± 66 |

Example 32

| | | |
|---|---|---|
| Cₘₐₓ (ng/mL) | 928 ± 372 | 316 ± 43 |
| tₘₐₓ (h) | 0.2 ± 0.1 | 0.25 ± 0 |
| AUC₀₋ₜ (ng * h/mL) | 544 ± 276 | 405 ± 41 |

Those skilled in the art will appreciate that numerous changes and modifications can be made to the preferred embodiments of the disclosure and that such changes and modifications can be made without departing from the spirit of the disclosure. It is, therefore, intended that the appended claims cover all such equivalent variations as fall within the true spirit and scope of the disclosure.

What is claimed:

1. A compound having the structure of formula (I), (II), (III), (IV), (V), (VI), (VII), or (VIII), or a pharmaceutically acceptable salt, stable isotope, or stereoisomer thereof: 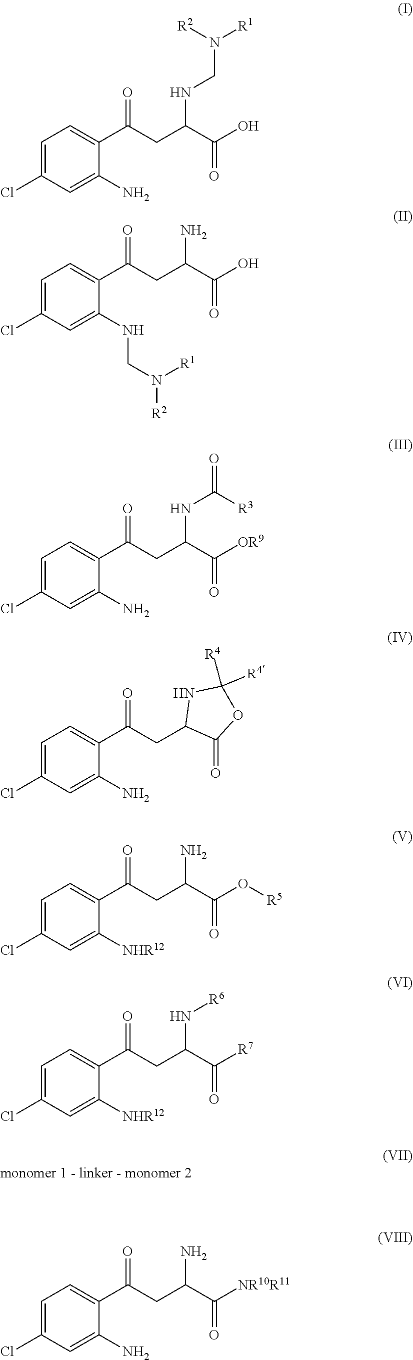
wherein:
R¹ and R² are, independently, optionally substituted C₁₋₆ alkyl, optionally substituted C₃₋₈ cycloalkyl, optionally substituted aryl, optionally substituted heteroaryl, or optionally substituted heterocyclyl; or
R¹ and R², together with the atoms to which they are attached, form an optionally substituted 4- to 8-membered heterocycle;
R³ is H, optionally substituted C₁₋₆ alkyl, optionally substituted C₁₋₆ alkoxy, optionally substituted arylC₁₋₆ alkyleneoxyl, optionally substituted C₃₋₈ cycloalkyl, optionally substituted aryl, —NH₂, —NHC₁₋₆ alkyl, —N(C₁₋₆ alkyl)₂, optionally substituted heteroaryl, or optionally substituted heterocyclyl;
R⁴ is H, optionally substituted C₁₋₆ alkyl, optionally substituted C₃₋₈ cycloalkyl, optionally substituted aryl, optionally substituted heteroaryl, or optionally substituted heterocyclyl;
R^{4′} is optionally substituted C₁₋₆ alkyl, optionally substituted C₃₋₈ cycloalkyl, optionally substituted aryl, optionally substituted heteroaryl, or optionally substituted heterocyclyl;
R⁵ is optionally substituted C₁₋₁₀ alkyl, optionally substituted aryl, optionally substituted alkylene glycol, —P(O)(OH)₂, —P(O)(OH)(OC₁₋₆alkyl), or —S(O)₂OH;
R⁶ is H, an amino acid moiety, or a peptide moiety;
R⁷ is OH, an amino acid moiety, or a peptide moiety;
wherein at least one of R⁶ and R⁷ is an amino acid moiety or a peptide moiety comprising at least 2 amino acid moieties; or
R⁸ is H or optionally substituted C₁₋₆ alkyl;
R⁹ is H or optionally substituted C₁₋₆ alkyl;
R¹⁰ and R¹¹ are, independently, H, optionally substituted C₁₋₆ alkyl, or SO₂(C₁₋₆ alkyl); or R¹⁰ and R¹¹, together with the atoms to which they are attached, form an optionally substituted heterocyclyl;
R¹² is H, C(O)C₁₋₆ alkyl, or C(O)OC₁₋₆ alkyl;
R¹³ is H; or R¹³ and R⁷ form a bond or CH₂ group;
linker is optionally substituted C₁₋₆ alkyl, optionally substituted C₃₋₈ cycloalkyl, optionally substituted aryl, optionally substituted heteroaryl, optionally substituted heterocyclyl, or optionally substituted glycol moiety; and
monomer 1 and monomer 2 are independently selected from the group consisting of a moiety of formula (I), (II), and (III);
wherein the compound converts to 4-chlorokynurenine after administration to a human.

2. The compound of claim 1 having the structure of formula (V): 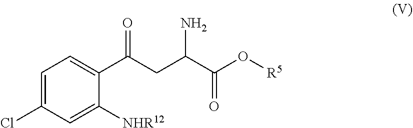
wherein:
R⁵ is optionally substituted C₁₋₁₀ alkyl, optionally substituted aryl, optionally substituted alkylene glycol, —P(O)(OH)₂, —P(O)(OH)(OC₁₋₆alkyl), or —S(O)₂OH; and
R¹² is H, C(O)C₁₋₆ alkyl, or C(O)OC₁₋₆ alkyl;
or a pharmaceutically acceptable salt, stable isotope, or stereoisomer thereof.

3. The compound of claim 2 having the structure of formula (VA), (VB), or (VC):

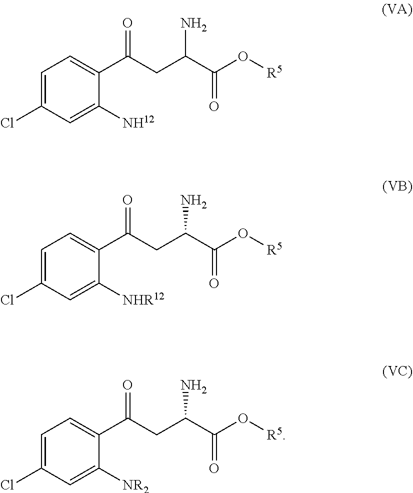

4. The compound of claim 2, wherein R⁵ is optionally substituted C₁₋₁₀ alkyl optionally substituted with optionally substituted aryl, C₁₋₁₀ alkyl substituted with optionally substituted heterocyclyl, or optionally substituted aryl.

5. The compound of claim 2, wherein R⁵ is alkylene glycol optionally substituted by C(O)aryl, C₁₋₆alkyl, phenyl, —P(O)(OH)₂, —P(O)(OH)(OC₁₋₆alkyl), or —S(O)₂OH.

6. The compound of claim 2, wherein R¹² is H.

7. The compound of claim 2, wherein R¹² is C₁₋₆ alkyl or C₁₋₆ alkoxy.

8. The compound of claim 1 having the structure of formula (III): 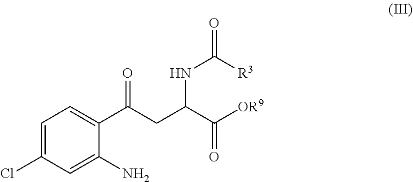
wherein:
R³ is H, optionally substituted C₁₋₆ alkyl, optionally substituted C₁₋₆ alkoxy, optionally substituted arylC₁₋₆ alkyleneoxyl, optionally substituted C₃₋₈ cycloalkyl, optionally substituted aryl, —NH₂, —NHC₁₋₆ alkyl, —N(C₁₋₆ alkyl)₂, optionally substituted heteroaryl, or optionally substituted heterocyclyl; and
R⁹ is H or optionally substituted C₁₋₆ alkyl;
or a pharmaceutically acceptable salt, stable isotope, or stereoisomer thereof.

9. The compound of claim 11, which has the structure of formula (IIIA), (IIIB), or (IIIC):

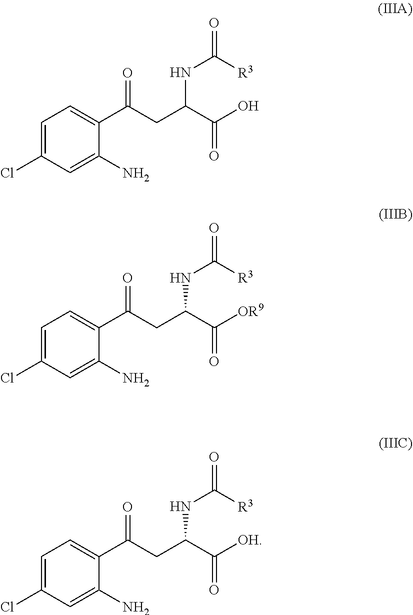

10. The compound of claim 11, wherein R³ is C₁₋₆ alkyl, C₁₋₆ alkoxy, or optionally substituted arylC₁₋₆ alkyleneoxyl.

11. The compound of claim 11, wherein R³ is optionally substituted C₃₋₈ cycloalkyl, optionally substituted aryl, optionally substituted heteroaryl, or optionally substituted heterocyclyl.

12. The compound of claim 11, wherein R³ is —NH₂, —NHC₁₋₆ alkyl, or —N(C₁₋₆alkyl)₂.

13. The compound of claim 11, wherein R⁹ is H or optionally substituted C₁₋₆ alkyl.

14. The compound of claim 1 having the structure of formula (VI): 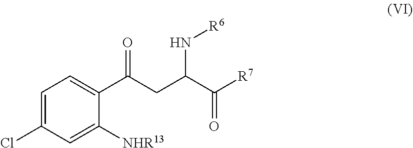
wherein:
R⁶ is H, an amino acid moiety, or a peptide moiety;
R⁷ is OH, an amino acid moiety, or a peptide moiety;
wherein at least one of R⁶ and R⁷ is an amino acid moiety or a peptide moiety comprising at least 2 amino acid moieties; or
R¹³ is H; or R¹³ and R⁷ form a bond or CH₂ group;
or a pharmaceutically acceptable salt, stable isotope, or stereoisomer thereof.

15. The compound of claim 18 having the structure of formula (VIA), (VIB), or (VIC):

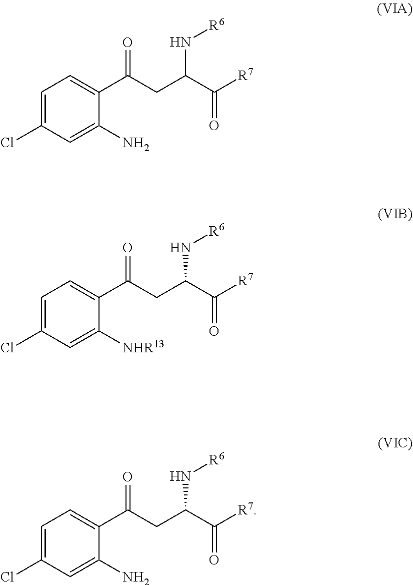

16. The compound of claim 18, wherein said peptide moiety comprises 2 to about 4 amino acids.

17. A compound that is

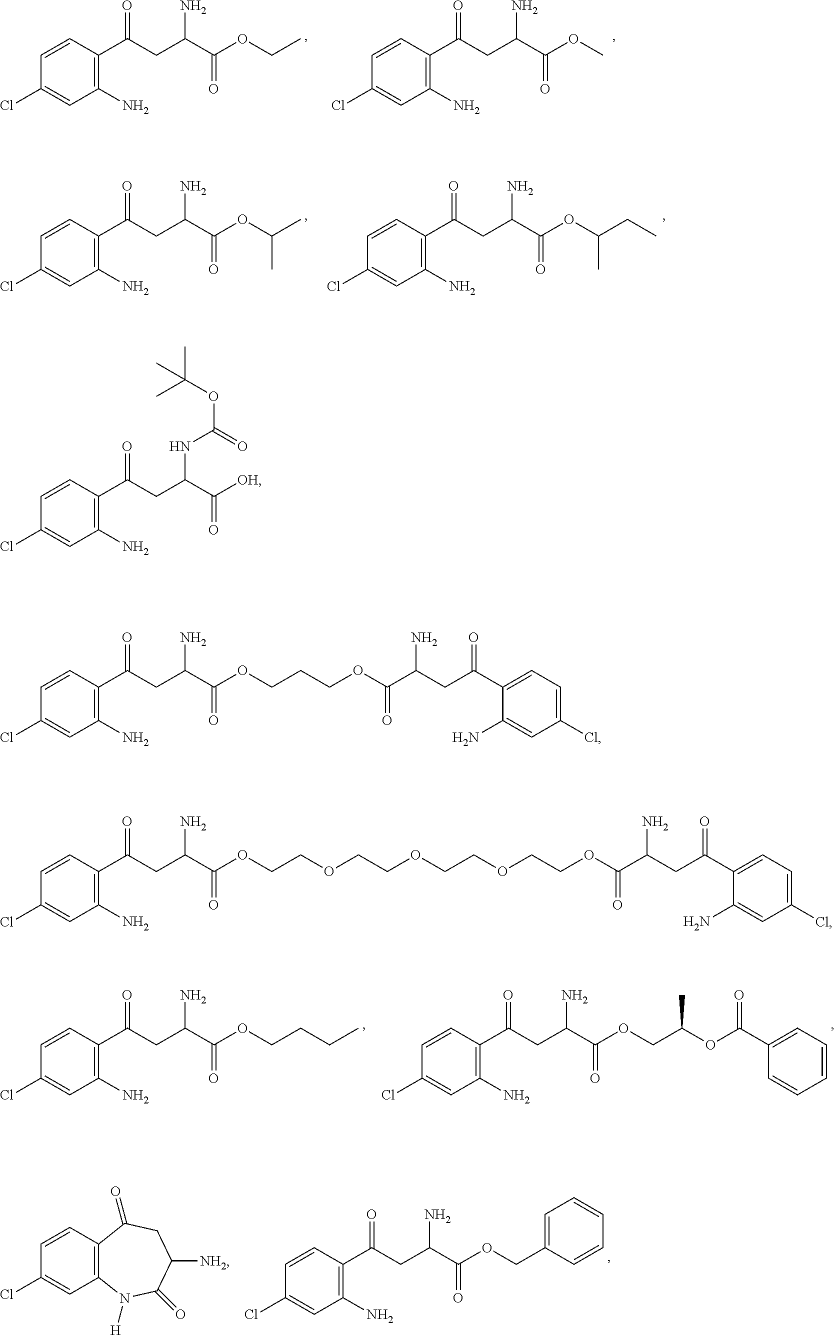

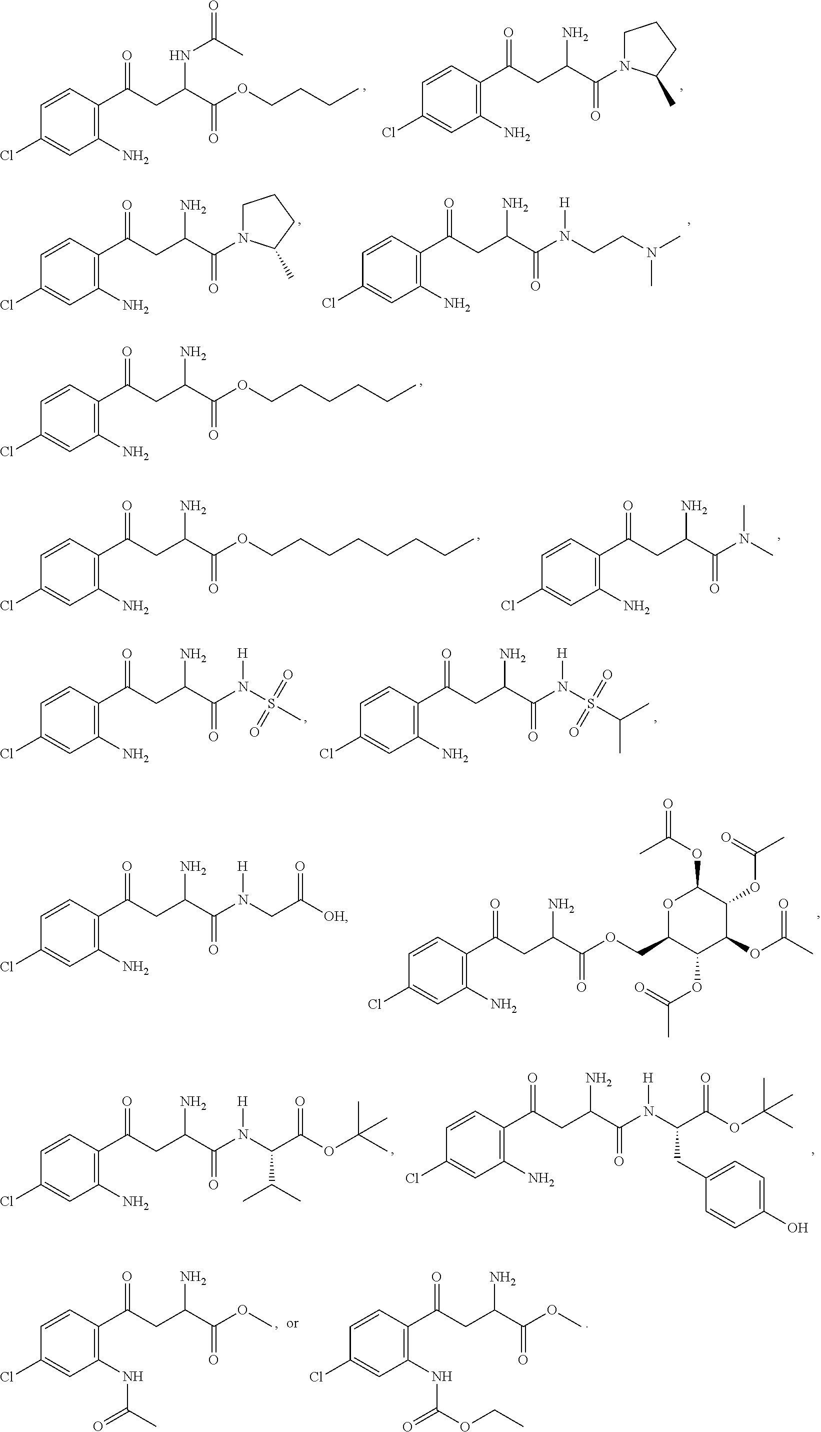

18. The compound of claim 1, wherein one or more H is replaced with ²H, one or more C is replaced with ¹³C, or one or more N is replaced with ¹⁵N.

19. A pharmaceutical composition comprising a compound of claim 1 and a pharmaceutically acceptable excipient.

20. A method of treating a neurodegenerative disorder, enhancing learning, memory, or cognition, treating a condition caused by neurological dysfunction, treating depression, treating hyperalgesia or reducing a L-DOPA associated dyskinesia in a patient comprising administering a compound of claim 1 to the patient.
